(19) United States
(12) Patent Application Publication
(10) Pub. No.: US 2015/0025127 A1
(43) Pub. Date: Jan. 22, 2015

(54) COMPOSITIONS AND METHODS FOR INDUCING APOPTOSIS

(71) Applicant: VIRXSYS CORPORATION, Gaithersburg, MD (US)

(72) Inventor: Gerard John McGarrity, Gaithersburg, MD (US)

(21) Appl. No.: 14/238,304

(22) PCT Filed: Aug. 10, 2012
(86) PCT No.: PCT/US12/50249

§ 371 Date: Sep. 25, 2014

Related U.S. Application Data
(60) Provisional application No. 61/522,844, filed on Aug. 12, 2011.

(51) Int. Cl. C12N 15/85

(52) U.S. Cl. CPC C12N 15/85
USPC 514/44 R; 536/23.1; 435/325; 435/455; 435/320.1

(57) ABSTRACT

Methods and compositions are provided for generating novel nucleic acid molecules through targeted spliceosome mediated RNA trans-splicing (SMaRT™) that result in expression of a splicing isoform or variant thereof. The methods and compositions are based upon pre-trans-splicing molecules (PTMs) designed to interact with a target pre-mRNA molecule and mediate a trans-splicing reaction generating a novel chimeric RNA molecule encoding a splicing isoform for the treatment of a variety of gene isoform induced diseases such as cancer.

### CROSS REFERENCE TO RELATED APPLICATIONS

This application claims the benefit of U.S. Provisional Patent Application No. 61/522,844, filed on Aug. 12, 2011, the disclosure of which is hereby incorporated by reference in its entirety.

### SEQUENCE LISTING

This application incorporates by reference the Sequence Listing contained in an ASCII text file named “356007-00146_ST25.txt” submitted via EFS-Web. The text file was created on Aug. 10, 2012, and is 1.57 kb.

### FIELD OF THE INVENTION

This application relates to methods and compositions for generating novel nucleic acid molecules through RNA trans-splicing that target precursor messenger RNA molecule (target pre-mRNA) and contain the coding sequence of a protein or polypeptide of interest. In particular, this application relates to methods and compositions for the inducement of apoptosis by spliceosome mediated RNA trans-splicing, and, more particularly, to methods and compositions comprising pre-trans-splicing molecules (PTMs) to express apoptosis inducing splicing isoforms via spliceosome mediated RNA trans-splicing (SMaRT™).

### BACKGROUND OF THE INVENTION

RNA Splicing

DNA sequences in the chromosome are transcribed into pre-mRNAs which contain coding regions (exons) and generally also contain intervening non-coding regions (introns). Introns are removed from pre-mRNAs in a precise process called pre-mRNA splicing (splicing) (Chow et al., 1977, Cell 12:1-8; and Berget, S. M. et al., 1977, Proc. Natl. Acad. Sci. USA 74:3171-3175). Splicing takes place as a coordinated interaction of several small nuclear ribonucleoprotein particles (snRNPs) and many protein factors that assemble to form an enzymatic complex known as the spliceosome (Moore et al., 1993, The RNA World, R. F. Gestland and J. F. Atkins eds. (Cold Spring Harbor Laboratory Press, Cold Spring Harbor, N.Y.); Kramer, 1996, Annu. Rev. Biochem., 65:367-404; Staley and Guthrie, 1998, Cell 92:315-326).

In most cases, the splicing reaction occurs within the same pre-mRNA molecule, which is termed cis-splicing. Splicing between two independently transcribed pre-mRNAs is termed trans-splicing. Trans-splicing was first discovered in trypanosomes (Sutton & Boothroyd, 1986, Cell 47:527; Murphy et al., 1986, Cell 47:517) and subsequently in nematodes (Krause & Hirsh, 1987, Cell 49:753); flatworms (Rajkovic et al., 1990, Proc. Natl. Acad. Sci. USA, 87:8879; Davis et al., 1995, J. Biol. Chem. 270:21813) and in plant mitochondria (Malek et al., 1997, Proc. Natl. Acad. Sci. USA 94:553). In the parasite Trypanosoma brucei, all mRNAs acquire a splice leader (SL) RNA at their 5′ termini by trans-splicing. A 5′ leader sequence is also trans-spliced onto some genes in Caenorhabditis elegans. This mechanism is appropriate for adding a single common sequence to many different transcripts.

The mechanism of splice leader trans-splicing, which is nearly identical to that of conventional cis-splicing, proceeds via two phosphoryl transfer reactions. The first causes the formation of a 2′-5′ phosphodiester bond producing a “Y” shaped branched intermediate, equivalent to the lariat intermediate in cis-splicing. The second reaction, exon ligation, proceeds as in conventional cis-splicing. In addition, sequences at the 3′ splice site and some of the snRNPs, which catalyze the trans-splicing reaction, closely resemble their counterparts involved in cis-splicing.

Trans-splicing between conventional pre-mRNAs refers to a different process, where an intron of one pre-mRNA interacts with an intron of a second pre-mRNA, enhancing the recombination of splice sites between two conventional pre-mRNAs. This type of trans-splicing was postulated to account for transcripts encoding a human immunoglobulin variable region sequence linked to the endogenous constant region in a transgenic mouse (Shimizu et al., 1989, Proc. Natl. Acad. Sci. USA 86:8020). In addition, trans-splicing of c-myb pre-mRNA has been demonstrated (Vellard, M. et al., 1992, Proc. Natl. Acad. Sci. USA 89:2511-2515) and RNA transcripts from cloned SV40 trans-spliced to each other were detected in cultured cells and nuclear extracts (Eul et al., 1995, EMBO. J 14:3226). However, naturally occurring trans-splicing of mammalian pre-mRNAs is thought to be a rare event (Flouriot G. et al., 2002, J. Biol. Chem: Finta, C. et al., 2002 J. Biol. Chem. 277:5882-5890).

In vitro trans-splicing has been used as a model system to examine the mechanism of splicing by several groups (Konarska & Sharp, 1985, Cell 46:165-171; Solnick, 1985, Cell 42:157; Chiara & Reed, 1995, Nature 375:510; Pasman and Garcia-Blanco, 1996, Nucleic Acids Res. 24:1638). Reasonably efficient trans-splicing (30% of cis-spliced analog) was achieved between RNAs capable of base pairing to each other, splicing of RNAs not tethered by base pairing was further diminished by a factor of 10. Other in vitro trans-splicing reactions not requiring obvious RNA-RNA interactions among the substrates were observed by Chiara & Reed (1995, Nature 375:510), Bruzik J. P. & Maniatis, T. (1992, Nature 360:692) and Bruzik J. P. and Maniatis, T., (1995, Proc. Natl. Acad. Sci. USA 92:7056-7059). These reactions yield very low levels of spliced products, and require specialized elements, such as a downstream 5′ splice site or exonic splicing enhancers.

In addition to splicing mechanisms involving the binding of multiple proteins to the pre-messenger RNA (mRNA) which then act to correctly cut and join RNA, a third mechanism involves cutting and joining of the RNA by the intron itself, by what are termed catalytic RNA molecules or ribozymes. The cleavage activity of ribozymes has been targeted to specific RNAs by engineering a discrete “hybridization” region into the ribozyme. Upon hybridization to the target RNA, the catalytic region of the ribozyme cleaves the target. It has been suggested that such ribozyme activity would be useful for the inactivation or cleavage of target RNA in vivo, such as for the treatment of human diseases characterized by production of foreign or aberrant RNA. In such instances, small RNA molecules are designed to hybridize to the target RNA and by binding to the target RNA prevent translation of the target RNA or cause destruction of the RNA through activation of nucleases. The use of antisense RNA has also been proposed as an alternative mechanism for targeting and destruction of specific RNAs. Others have attempted to select one from different spliceoforms. This has been reviewed by Kole and colleagues in the area of this invention, namely to inactivate an anti-apoptotic isoform of Bx-L and convert it into a pro-apoptotic isoform, Bx-S. Bauman and Kole explored the use of splice switching oligonucleotides (SSO) or anti-sense by directing pre-mRNA splice site usage. Redirection of Bcl-x pre-mRNA splicing from Bcl-XL to Bcl-XS by SSO induced apoptosis and chemosensitivity effective in cancer cell lines. (Bioengineered Bugs 2:125-128, 2011). While antisense offers possible applications, there is a great specificity of the antisense molecule employed. A relatively minor alteration in the antisense molecule or the target can completely negate any positive effect on the target. This separates antisense from the present inventor's technology, RNA trans-splicing.

Using the Tetrahymena group I ribozyme, targeted trans-splicing was demonstrated in E. coli (Sullenger B. A. and Cech. T. R., 1994, Nature 341:619-622), in mouse fibroblasts (Jones, J. T. et al., 1996, Nature Medicine 2:643-648), human fibroblasts (Phylacton, L. A. et al., Nature Genetics 18:378-381 (1998)) and human erythroid precursors (Lan et al., 1998, Science 280:1593-1596). For a review of clinically relevant technologies to modify RNA see Sullenger and Gilboa, 2002 Nature 418:252-8.

Alternative Splicing and Human Disease

Alternative splicing is the major source of proteome diversity in humans and thus is thought to be highly relevant to human disease and therapy. Several important diseases have been linked to mutations or variations in either cis-splicing elements or certain trans-acting factors that lead to aberrant splicing and concomitant abnormal protein production (Garcia-Blanco et al., 2004, Nature Biotechnology 22 (535-546)). Alternative splicing is the process by which a single primary transcript yields different mature mRNAs leading to the production of protein isoforms with diverse and even antagonistic functions. Annotation of the human genome has revealed that the bulk of intron-containing transcripts are alternatively spliced. It is estimated that 95% of pre-mRNAs are alternatively spliced. The involvement of aberrant splicing in human disease has been recently reviewed (Ward and Cooper, J. Pathol. 2010 January; 220(2):152-63; Cooper, A. Wan L, Dreyfuss G. Cell. 2009 Feb. 20; 136(4):777-93; Orengo J P, Cooper T. A. Adv Exp Med Biol. 2007, 623:212-23; Wang G. S., Cooper T. A. Nat. Rev. Genet. 2007 October; 8(10):749-61. Epub 2007 Aug. 29). The role and extent of alternative splicing is reviewed in Nilsen T. W., Graveley B. R. Nature 2010 Jan. 28;463(7280):457-63. Computational biologists grapple with RNA's complexity (Ledford H., 2010, Nature 465:16-17, 2010). Although it was quickly recognized how extensive alternate splicing was, no one could predict which form would be expressed in different tissues.

Primary transcripts of complex protein-coding genes contain introns that must be removed by the splicing apparatus. The efficiency and capacity of this apparatus are underscored by calculations of the number of introns that need to be removed at any one time and the speed with which the introns themselves must be removed (Garcia-Blanco et al., 2004, Nature Biotechnology 22 (535-546)). RNA splicing depends on the proper recognition of exons, the usual size of which is 300 nucleotides for terminal exons with the average internal exon being only 145 nucleotides in length. There are six known different types of alternative splicing. In rare cases, an entire intron is removed or retained to result in two very different RNAs. Alternative 5′ splice sites or 3′ splice sites can result in exons of different size. Inclusion or skipping of one or more exons is a common form of alternative splicing. Alternative splicing of transcripts initiated at different transcription start sites leads to mature RNAs with different first exons. The 3′ terminal exons can also vary by coupling alternative splicing with alternative polyadenylation. Finally, a rare form of alternative splicing involves reactions between two primary transcripts in trans (Garcia-Blanco et al., 2004, Nature Biotechnology 22 (535-546).

The splicing patterns of several genes have been reported to be altered in cancers, including those encoding the prolactin receptor, Ron, Rac1, fibronectin, fibroblast growth factor receptors, CD44, MDM2, and IIp45 (Srebrow, A. and Kornblihtt, A. R., 2006, J Cell Sci 119:2635-2641). Certain alternatively spliced isoforms of proteins such as Ron and Rac1 can accumulate in tumors, and overexpression of the tumor-associated isoforms is sufficient to transform cells in culture (Singh et al., 2004, Oncogene 23:9369-9380); Zhou et al., 2003, Oncogene 22:186-197).

The up regulation of particular splice isoforms in preference to others has been implicated in several cancers. The apoptotic regulator Bcl-X is one example where two isoforms have opposing effects on apoptosis (Boise, L. H. et al., 1993, Cell 74:597-608). Bcl-X_{S} is pro-apoptotic while Bcl-X_{L} is anti-apoptotic. This difference in function depends on use of an alternative 5′-splice site in the first coding exon.

It is therefore clear that alternative splice variants, which may be tumor specific, can significantly influence cellular processes in cancer, including cell proliferation, motility and chemosensitivity or drug response. However, the degree to which aberrant splicing is involved in carcinogenesis and how much is merely a reflection of the generally disordered cell processes present in tumors, remains largely uncertain.

Accordingly, a continuing and unmet need exists for new, improved, safer, and alternative means for modulating RNA splicing as a potential treatment for those diseases or disorders that are mediated by alternative RNA splicing isoforms. This invention addresses these and other needs by the use of trans-splicing to induce the expression of disease-ameliorating gene splicing isoforms in general, and to induce the expression of apoptosis splicing isoforms in particular, so as to induce a non-apoptotic cell into an apoptotic state as a means to treat cancer directly and/or render a cancer cell more susceptible to other cancer therapeutics.

Citation of the above documents or any references cited herein is not intended as an admission that any of the foregoing is pertinent prior art. All statements as to the date or representation as to the contents of these documents is based on the information available to the inventor and does not constitute any admission as to the correctness of the dates or contents of these documents.

### SUMMARY OF THE INVENTION

The present invention relates to compositions and methods for generating novel nucleic acid molecules through spliceosome-mediated targeted RNA trans-splicing (SMaRT™). The compositions of the invention include pre-trans-splicing molecules (hereinafter referred to as “PTMs”) expressing a splicing isoform designed to interact with a natural target pre-mRNA molecule (hereinafter referred to as “pre-mRNA”) and mediate a spliceosomal trans-splicing reaction resulting in the generation of a novel chimeric RNA molecule (hereinafter referred to as “chimeric RNA”). The methods of the invention encompass contacting a splicing isoform PTM of the invention with a natural target pre-mRNA under conditions in which all or portion of the splicing isoform PTM is spliced to the natural pre-mRNA to form a novel chimeric RNA. Generally, the target pre-mRNA is chosen because it is expressed within a specific cell type (for example, a cell type expressing the disease-causing splicing isoform) thereby providing a means for targeting expression of the novel chimeric RNA to a selected cell type, for example, and not by way of limitation, a cancer cell.

In particular, the compositions of the present invention include nucleic acid molecules containing at least one PTM expressing an apoptosis inducing splicing isoform which, upon trans-splicing using SMaRT™ to a target pre-mRNA expressed within the cell, produce a splicing isoform that drives a non-apoptotic cell into apoptosis.

In one embodiment of the present invention, a nucleic acid molecule is provided that encodes an apoptosis inducing splicing isoform wherein said nucleic acid molecule comprises: a) one or more target binding domains that target binding of the nucleic acid molecule that encodes the apoptosis inducing splicing isoform to a target pre-mRNA expressed within the cell, wherein said nucleic acid molecule is recognized by nuclear splicing components within the cell; and wherein said isolated nucleic acid molecule encodes an apoptosis inducing splicing isoform heterologous to the target pre-mRNA.

In another embodiment of the present invention, the apoptosis inducing splicing isoform PTMs further comprise one or more target binding domains that target binding of the PTM to an endogenous heterologous pre-mRNA; a 3′ splice region that includes a branch point, pyrimidine tract and a 3′ splice acceptor site, and/or a 5′ splice donor site; a spacer region to separate the RNA splice site from the target binding domain; and a safety sequence comprising one or more complementary sequences that bind to one or both sides of the 5′ splice site, or any combination thereof.

In another embodiment of the present invention, the compositions of the present invention include nucleic acid molecules comprising at least one PTM expressing an apoptosis inducing splicing isoform, wherein said nucleic acid molecule is recognized by nuclear splicing components within the cell, and designed to interact with an abundantly expressed target pre-mRNA molecule (including, for example, albumin, apoA-1, casein, actin, tubulin, myosin and fibroin) expressed within the cell which, upon trans-splicing using SMaRT™, produce a novel chimeric RNA molecule expressing a genetic splicing isoform that drives a non-apoptotic cell into apoptosis.

In another embodiment of the present invention, the apoptosis inducing splicing isoform PTMs further comprise one or more target binding domains that target binding of the PTM to an endogenous highly expressed heterologous pre-mRNA molecule (including, for example, albumin, apoA-1, casein, actin, tubulin, myosin and fibroin); a 3′ splice region that includes a branch point, pyrimidine tract and a 3′ splice acceptor site, and/or a 5′ splice donor site; a spacer region to separate the RNA splice site from the target binding domain; and a safety sequence comprising one or more complementary sequences that bind to one or both sides of the 5′ splice site, or any combination thereof.

In another aspect of the present invention, a cell is provided comprising at least one PTM expressing an apoptosis inducing splicing isoform wherein said nucleic acid molecule is recognized by nuclear splicing components within the cell, and designed to interact with a target heterologous pre-mRNA or an abundantly expressed heterologous target pre-mRNA molecule (including, for example, albumin, casein, actin, tubulin, myosin and fibroin) expressed within the cell which, upon trans-splicing using SMaRT™, produces a novel chimeric RNA molecule expressing a genetic splicing isoform that drives a non-apoptotic cell into apoptosis.

In another embodiment of the cell of the present invention, the apoptosis inducing splicing isoform PTMs further comprise one or more target binding domains that target binding of the PTM to an endogenous heterologous pre-mRNA; a 3′ splice region that includes a branch point, pyrimidine tract and a 3′ splice acceptor site, and/or a 5′ splice donor site; a spacer region to separate the RNA splice site from the target binding domain; and a safety sequence comprising one or more complementary sequences that bind to one or both sides of the 5′ splice site, or any combination thereof.

In ones embodiment of the present invention, the cell is a non-apoptotic cancerous cell comprising for example, and not by way of limitation, a cancer cell associated with multiple myeloma, small cell lung cancer, prostate and breast cancer or said cancer may be selected from the group consisting of breast, glioma, large intestinal cancer, lung cancer, small cell lung cancer, stomach cancer, liver cancer, blood cancer, bone cancer, pancreatic cancer, skin cancer, head and neck cancer, cutaneous or intraocular melanoma, uterine sarcoma, ovarian cancer, rectal or colorectal cancer, anal cancer, colon cancer, fallopian tube carcinoma, endometrial carcinoma, cervical cancer, vulval cancer, squamous cell carcinoma, vaginal carcinoma, Hodgkin's disease, non-Hodgkin's lymphoma, multiple myeloma, esophageal cancer, small intestine cancer, endocrine cancer, thyroid cancer, parathyroid cancer, adrenal cancer, soft tissue tumor, urethral cancer, penile cancer, prostate cancer, chronic or acute leukemia, lymphocytic lymphoma, bladder cancer, kidney cancer, ureter cancer, renal cell carcinoma, renal pelvic carcinoma, CNS tumor, glioma, astrocytoma, glioblastoma multiforme, primary CNS lymphoma, bone marrow tumor, brain stem nerve gliomas, pituitary adenoma, uveal melanoma, testicular cancer, oral cancer, pharyngeal cancer, pediatric neoplasms, leukemia, neuroblastoma, retinoblastoma, glioma, rhabdomyoblastoma and sarcoma.

In another embodiment of the present invention, an expression vector is provided wherein said vector expresses a nucleic acid molecule comprising at least one PTM expressing an apoptosis inducing splicing isoform, and wherein said nucleic acid molecule further comprises a) one or more target binding domains that target binding of the nucleic acid molecule to a non-apoptosis inducing splicing isoform target pre-mRNA expressed within a cell; b) a 3′ splice region comprising a branch point, a pyrimidine tract and a 3′ splice acceptor site; c) a spacer region that separates the 3′ splice region from the target binding domain; and d) a nucleotide sequence to be trans-spliced to the target pre-mRNA; wherein said nucleic acid molecule is recognized by nuclear splicing components within the cell.

In another embodiment of the present invention, an expression vector is provided wherein said vector expresses a nucleic acid molecule comprising at least one PTM expressing an apoptosis inducing splicing isoform, and wherein said nucleic acid molecule further comprises a) one or more target binding domains that target binding of the nucleic acid molecule to a non-apoptosis inducing splicing isoform target pre-mRNA expressed within a cell; b) a 5′ splice site; c) a spacer region that separates the 5′ splice site from the target binding domain; and d) a nucleotide sequence to be trans-spliced to the target pre-mRNA; wherein said nucleic acid molecule is recognized by nuclear splicing components within the cell.

In each of the aforementioned embodiments of the present invention, the apoptosis inducing splicing isoform comprises an apoptosis inducing splicing isoform gene product of a Bcl family gene, an FGFR2 family gene, p53 a family gene, an RAD51, a survivin family gene (survivin and survivin 2-B), a Bim family gene, a Bcl-2 family gene, an Apa F-1 family gene, a procaspase family gene, an Fas family gene, an Rb family gene, or any one of the known or hereafter discovered apoptosis inducing splicing isoforms, or any combination thereof.

In yet another aspect of the present invention, a method is provided for driving a non-apoptotic cell into apoptosis comprising introducing into a non-apoptotic cell at least one PTM encoding a splicing isoform wherein said nucleic acid molecule is recognized by nuclear splicing components within the cell; trans-splicing said at least one PTM encoding a splicing isoform into an endogenous heterologous pre-mRNA using SMaRT™; wherein trans-splicing of at least one PTM encoding a splicing isoform into an endogenous heterologous pre-mRNA produces a functional transcript which is then translated into a splicing isoform that induces the non-apoptotic cell into an apoptotic cell.

In another embodiment of the present invention, the method further comprises the step of targeting binding of said PTM, wherein said PTM comprises one or more target binding domains that target binding of the PTM to an endogenous heterologous pre-mRNA of the cell; a 3′ splice region that includes a branch point, pyrimidine tract and a 3′ splice acceptor site and/or 5′ splice donor site; a spacer region to separate the RNA splice site from the target binding domain; and a safety sequence comprising one or more complementary sequences that bind to one or both sides of the 5′ splice site, or any combination thereof.

In yet another embodiment of the present invention a method is provided for producing a chimeric RNA molecule in a non-apoptotic cell comprising contacting a target pre-mRNA expressed in the cell with a nucleic acid molecule recognized by nuclear splicing components wherein said nucleic acid molecule comprises (a) one or more target binding domains that target binding of the nucleic acid molecule to a target heterologous pre-mRNA expressed within the cell, wherein said target binding domain targets a human albumin pre-mRNA; (b) a 3′ splice region comprising a branch point and a 3′ splice acceptor site; (c) a spacer region that separates the 3′ splice region from the target binding domain; and (d) a nucleotide sequence to be trans-spliced to the target pre-mRNA wherein said nucleotide sequence encodes an apoptosis inducing splicing isoform; under conditions in which a portion of the nucleic acid molecule is trans-spliced to a portion of the target heterologous pre-mRNA to form a chimeric RNA within the cell, and wherein the splicing isoform induces the non-apoptotic cell into an apoptotic cell.

In one embodiment of the methods of the present invention, the splicing isoform comprises at least one apoptosis inducing splicing isoform.

In another embodiment of the methods of the present invention, the apoptosis inducing splicing isoform comprises an apoptosis inducing splicing isoform gene product of a Bcl family gene, an FGFR2 family gene, p53 a family gene, an RAD51, a survivin family gene (survivin and survivin 2-B), a Bim family gene, an Apa F-1 family gene, an Mcl1 family gene, a caspase 2L family gene, a caspase-9 family gene, a procaspase family gene, a Fas family gene, a Herstatin family gene, a Δ15HER2 family gene, a Rac1 family gene, a VGEF165b family gene, a KLF6 family gene, an Rb family gene, any combination thereof.

In yet another embodiment of the methods of the present invention, the apoptosis inducing splicing isoform comprises at least one of Bcl Xₛ, Mcl-1S, Caspase-2L, Caspase-9, Survivin-2B, Fas, Herstatin, Δ15HER2, Rac1, VEGF165b, p53, KLF6, and RBM5, or any combination thereof.

In one embodiment of the methods of the present invention, the cell is a non-apoptotic cancerous cell comprising for example, and not by way of limitation, a cancer cell associated with multiple myeloma, small cell lung cancer, prostate and breast cancer said cancer is selected from the group consisting of breast, glioma, large intestinal cancer, lung cancer, small cell lung cancer, stomach cancer, liver cancer, blood cancer, bone cancer, pancreatic cancer, skin cancer, head and neck cancer, cutaneous or intraocular melanoma, uterine sarcoma, ovarian cancer, rectal or colorectal cancer, anal cancer, colon cancer, fallopian tube carcinoma, endometrial carcinoma, cervical cancer, vulval cancer, squamous cell carcinoma, vaginal carcinoma, Hodgkin's disease, non-Hodgkin's lymphoma, multiple myeloma, esophageal cancer, small intestine cancer, endocrine cancer, thyroid cancer, parathyroid cancer, adrenal cancer, soft tissue tumor, urethral cancer, penile cancer, prostate cancer, chronic or acute leukemia, lymphocytic lymphoma, bladder cancer, kidney cancer, ureter cancer, renal cell carcinoma, renal pelvic carcinoma, CNS tumor, glioma, astrocytoma, glioblastoma multiforme, primary CNS lymphoma, bone marrow tumor, brain stem nerve gliomas, pituitary adenoma, uveal melanoma, testicular cancer, oral cancer, pharyngeal cancer, pediatric neoplasms, leukemia, neuroblastoma, retinoblastoma, glioma, rhabdomyoblastoma and sarcoma.

In another embodiment of the methods of the present invention, the cell is a cancer cell expressing a non-apoptosis inducing splicing isoform including, for example, and not by way of limitation, Bcl-X_{L} or a functional derivative thereof.

In one embodiment of the methods of the present invention, the cell is a cancer cell that does not express a non-apoptosis inducing splicing isoform including, for example, and not by way of limitation, Bcl-X_{L} or a functional derivative thereof.

For each of the aforementioned embodiments of the compositions and methods of the present invention, without intended to be limited by any particular mechanism of action, it is believed that the induction of the Bcl X_{S} splicing isoform causes apoptosis by antagonizing the production of the Bcl-X_{L} splicing isoform, antagonizing the production of Bcl-2, causing a significant reduction in tumor load or of tumor burden in the cancer or cancerous tissue, or by increasing the sensitivity of the cancer or cancerous tissue to chemotherapeutic drugs, or any combination thereof.

For each of the aforementioned embodiments of the compositions and methods of the present invention, if so desired, for those embodiments utilizing a highly abundant or expressed pre-mRNA, cytoplasmic targeting of the splicing isoform may be achieved with, for example, and not by way of limitation, i) targeting of the PTM to cytoplasmically abundant or highly expressed proteins such as tubulin (exon 1) or actin (exon 2) or ii) the leader sequence of the protein encoded by the highly abundant or expressed pre-mRNA may be modified by inclusion of either a transmembrane anchoring domain (for example, a CD8 transmembrane domain corresponding to amino acids 137-212, as described in Santos, E B et al. Nature Med 15:338-344 March 2009, for a cytoplasmic anchoring domain known to those of skill in the art so as to ensure that the chimeric RNA resulting from the trans-splicing reaction produces the desired transmembrane protein or cytoplasmic protein.

In each of the aforementioned embodiments of the methods of the present invention, the trans-splicing is mediated by SMaRT. In another embodiment, the trans-splicing is mediated by Group I ribozymes. In yet another embodiment, the trans-splicing is mediated by Group II ribozymes.

The general design, construction and genetic engineering of PTMs and demonstration of their ability to successfully mediate trans-splicing reactions within the cell are described in detail in U.S. Pat. Nos. 6,083,702, 6,013,487, and 6,280,978, as well as patent application Ser. Nos. 09/756,095, 09/756,096, 09/756,097 09/941,492, US Patent Publication Nos. US 2006-0234247 A1, and US 2006-0194317 A1, the disclosures of each of which are incorporated herein by reference in their entirety.

The general design, construction and genetic engineering of trans-splicing ribozymes and demonstration of their ability to successfully mediate trans-splicing reactions within the cell are described in detail in and U.S. Pat. Nos. 5,667,969, 5,854,038 and 5,869,254, as well as patent application No. 20030036517, the disclosures of each of which are incorporated herein by reference in their entirety.

The design, construction and genetic engineering of PTMs expressing apoA-1 or other apoA-1 variants, and highly abundant expressed pre-RNA molecules are described in detail in U.S. Pat. Nos. 7,968,334 and 7,871,795, respectively, the disclosures of each of which are incorporated herein by reference in their entirety.

In one embodiment, for each of the aforementioned compositions and methods of the present invention, the PTMs expressing the apoptosis inducing splicing isoform are introduced into the cells using, for example, and not by way of limitation, retroviral vectors, lentiviral vectors, adeno-associated viral vectors, adenoviral vectors, pox virus vectors, cosmids, artificial chromosomes (e.g., YACs), plasmid/minicircle vectors, or any combination thereof, with the vectors themselves being delivered through electroporation, transformation, transduction, conjugation, transfection, infection, membrane fusion with cationic lipids, viral vector transduction, high-velocity bombardment with DNA-coated microprojectiles, incubation with calcium phosphate-DNA precipitate, or direct microinjection into single cells, or any combination thereof.

In each of the aforementioned embodiments of the compositions and methods of the present invention, the expression of the PTMs can be regulated by a constitutive promoter(s) or an inducible promoter(s) or a tissue specific promoter(s) or their combination, and may be bidirectional, capable of driving the expression of one or more different PTMs in a single vector. In certain embodiments of the present invention, the heterologous promoter comprises viral, human, and/or synthetic promoters or a combination thereof. In one embodiment, heterologous viral promoters comprise Mouse Mammary Tumor Virus (MMTV) promoter, Moloney virus, avian leukosis virus (ALV), Cytomegalovirus (CMV) immediate early promoter/enhancer, Rous Sarcoma Virus (RSV), adeno-associated virus (AAV) promoters; adenoviral promoters, and Epstein Barr Virus (EBV) promoters, lentiviral promoters, or any combination thereof. In another embodiment, heterologous human promoters comprise Apolipoprotein E promoter, Albumin promoter, Human ubiquitin C promoter, human tissue specific promoters such as liver specific promoter (for example, HCR-hATT), prostate specific antigen (PSA) promoter, Human phosphoglycerate kinase (PGK) promoter, Elongation factor-1 alpha (EF-1a) promoter, dectin-2 promoter, HLA-DR promoter, Human CD4 (hCD4) promoter, or any combination thereof. In yet another embodiment, the synthetic promoters comprise those promoters described in U.S. Pat. No. 6,072,050, the contents of which are incorporated by reference in their entirety.

For each of the aforementioned embodiments, the compositions and methods of the present invention can comprise the apoptosis inducing splicing isoform PTMs that target a highly abundant or expressed pre-mRNAs such as, for example, and not by way of limitation, casein, myosin and fibroin, tumor-specific or tumor associated transcripts, microbial or autoantigen associated transcripts, viral or yeast associated transcripts.

In one additional aspect of the present invention, for each of the aforementioned embodiments, the compositions and methods of the present invention can comprise splicing isoform PTMs that specifically target a splicing isoform directly responsible for a disease or condition, with the expressly intended negative limitation-based proviso that i) such splicing isoform expressly excludes the Tau isoform (and those functional derivatives thereof) responsible for any disease indication in general (for example, and not by way of limitation, Alzheimer disease, Nieman-Pick disease, progressive supranuclear palsy, and corticobasal degeneration), or the Tau isoform (and those functional derivatives thereof) responsible for fronto-temporal dementia with parkinsonism in particular, which Tau isoform is linked to chromosome 17 (FTDP-17), which is caused by a mutation in the MAPT gene encoding the tau protein that accumulates in intraneuronal lesions in a number of neurogenerative diseases (Rodriguez-Martin et al., 2009, Human Mol. Genet. 18: 3266-3273); and ii) the PTM-based invention disclosed herein expressly excludes the use of SMaRT to treat cancer or genetic, autoimmune or infectious diseases using a PTM expressing a suicide gene including, for example, the cell death inducing Diptheria toxin or subunit thereof as described in U.S. Pat. No. 6,013,487.

In another aspect of the present invention, each of the aforementioned compositions of the present invention may be formulated in a physiologically acceptable carrier.

These and other aspects of some exemplary embodiments will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments without departing from the spirit thereof. Additional features may be understood by referring to the accompanying drawings, which should be read in conjunction with the following detailed description and examples.

### BRIEF DESCRIPTION OF THE DRAWINGS

FIG. 1 schematically illustrates the use of SMaRT™ to mediate trans-splicing of a Bcl X splicing isoform. FIG. 1A depicts an example of 3′ exon replacement. FIG. 1B depicts an example of 5′ exon replacement. FIG. 1C depicts an example of internal exon replacement through double trans-splicing.

FIG. 2 schematically illustrates a lentiviral vector expressing a Bcl X gene isoform PTM. FIG. 2A depicts a schematic diagram of a lentiviral vector expressing a 3′ Bcl Xₛ gene isoform PTM. FIG. 2B depicts a schematic diagram of a lentiviral vector expressing a 5′ Bcl Xₛ gene isoform PTM. FIG. 2C depicts a schematic diagram of a lentiviral vector expressing a double trans-splicing Bcl Xₛ gene isoform PTM.

FIG. 3 schematically illustrates an example of targeting highly abundant transcripts such as albumin pre-mRNA target and production of Bcl-Xₛ pro-apoptotic mRNA using a 3′ PTM.

The diagrams illustrated in the drawings are not drawn to scale, and the relative sizes of particular segments or functional elements are not necessarily proportional to the lengths (e.g., number of base pairs) of the corresponding sequences.

### DETAILED DESCRIPTION OF THE INVENTION

PTMs Encoding Apoptosis Inducing Splicing isoform or Variants Thereof

The present invention relates to compositions and methods for generating novel nucleic acid molecules through spliceosome-mediated targeted RNA trans-splicing. The compositions of the invention include apoptosis inducing splicing isoform pre-trans-splicing molecules (PTMs) designed to interact with a natural target pre-mRNA molecule (pre-mRNA) and mediate a spliceosomal trans-splicing reaction resulting in the generation of a novel chimeric RNA molecule (chimeric RNA). The methods of the invention encompass contacting the apoptosis inducing splicing isoform PTMs of the invention with a natural target pre-mRNA under conditions in which a portion of the apoptosis inducing splicing isoform PTM is spliced to the natural pre-mRNA to form a novel chimeric RNA. The apoptosis inducing splicing isoform PTMs of the invention are genetically engineered so that the novel chimeric RNA resulting from the trans-splicing reaction may encode a protein that provides health benefits. Generally, the target pre-mRNA is chosen because it is expressed within a specific cell type thereby providing a means for targeting expression of the novel chimeric RNA to a selected cell type. For example, the apoptosis inducing splicing isoform PTMs may be targeted to abundantly expressed pre-mRNAs expressed in the liver such as albumin pre-mRNA.

In each embodiment of the compositions of the aforementioned apoptosis inducing splicing isoform PTMs of the present invention and methods of using same as described in detail herein, the apoptosis inducing splicing isoform encoded by the at least one PTM also specifically includes those derivatives, fragments or modifications thereof, which upon trans-splicing, cause expression of apoptosis inducing splicing isoform or convert other highly abundant expressed proteins such as albumin to produce apoptosis inducing splicing isoform function. Any and all such nucleotide variations and resulting amino acid polymorphisms or variations of the apoptosis inducing splicing isoform PTMs described herein that are the result of natural genotypic, allelic variation, or that have been artificially engineered, and which, upon trans-splicing, cause expression of apoptosis inducing splicing isoform or convert other highly abundant expressed proteins such as albumin to produce apoptosis inducing splicing isoform function, are intended to be within the scope of the invention.

Thus, derivatives, fragments or modifications thereof of the apoptosis inducing splicing isoform encoded by the at least one PTM can be created by introducing one or more nucleotide substitutions, additions or deletions into the nucleotide sequence of the apoptosis inducing splicing isoform, such that one or more amino acid residue substitutions, additions, or deletions are introduced into the apoptosis inducing splicing isoform encoded by the at least one PTM. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence of the apoptosis inducing splicing isoform PTMs, such as by saturation mutagenesis, and the resultant mutants can be screened for biological activity to identify mutants that, upon trans-splicing using SMaRT™, cause expression of apoptosis inducing splicing isoform or convert other highly abundant expressed proteins such as albumin to produce apoptosis inducing splicing isoform function.

The PTMs coding for apoptosis inducing splicing isoform are introduced into the cells using, for example, and not by way of limitation, retroviral vectors, lentiviral vectors, adeno-associated viral based vectors, adenoviral vectors, pox virus vectors, plasmid/minicircle vector, viral vector transduction, electroporation, transformation, transduction, conjugation, transfection, infection, membrane fusion with cationic lipids, high-velocity bombardment with DNA-coated microprojectiles, incubation with calcium phosphate-DNA precipitate, or direct microinjection into single cells. The apoptosis inducing splicing isoform PTM is targeted to endogenous pre-mRNAs that are expressed in the dividing or non-dividing somatic cells, and following trans-splicing, cause expression of apoptosis inducing splicing isoform or convert other highly abundant expressed proteins such as albumin to produce apoptosis inducing splicing isoform function.

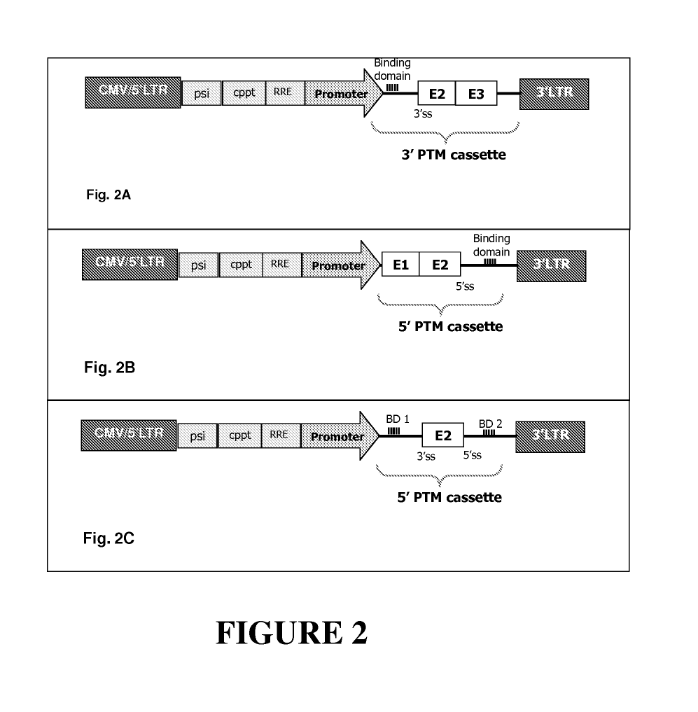

In another embodiment, for example, a lentiviral vector (a SIN-based or an integrase-deficient lentiviral vector as described more particularly infra) may be used to express the PTMs coding for an apoptosis inducing splicing isoform as depicted in FIG. 2. In yet another embodiment, an adenoviral associated vector such as for example, AAV serotypes 1-11 may be used to express the PTMs coding for an apoptosis inducing splicing isoform. Alternatively, in those instances, for example, when specific tissues are targeted such as hepatocytes, a chimeric adeno-associated vector such as AAV-DJ (Grim et al., 2008, J. Virol. 82: 5887, the entire contents of which are incorporated here by reference) may be used to express the PTMs coding for an apoptosis inducing splicing isoform. In yet other embodiments, lentiviral-adeno associated hybrid vectors more particularly described in Applicant's co-pending International Patent Application No. PCT/US2009/054378 (the contents of which are incorporated herein by reference in their entirety) may be used to the PTMs coding for an apoptosis inducing splicing isoform.

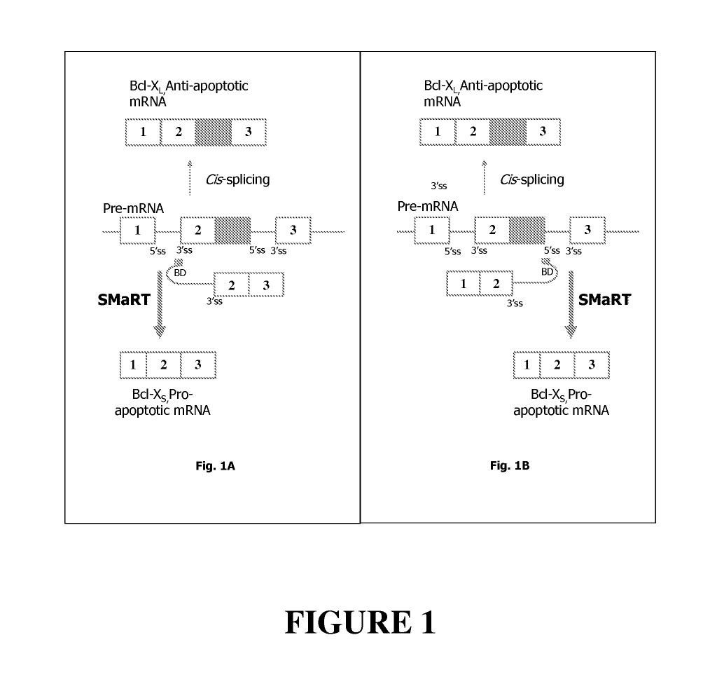

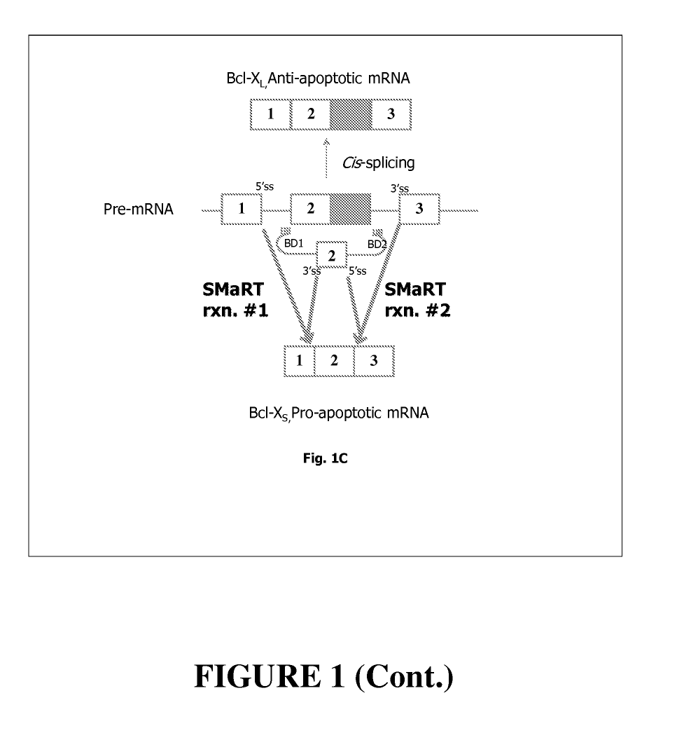

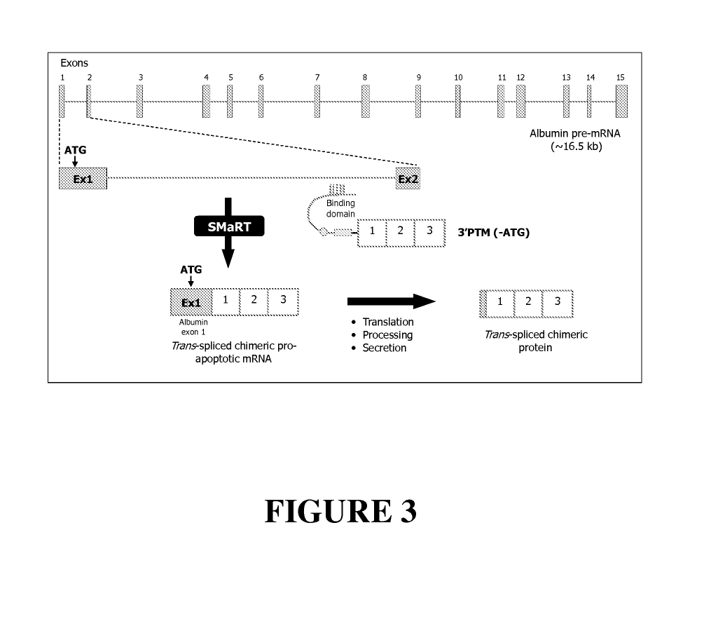

Now referring specifically to the attached drawings, in one embodiment of the present invention, a schematic representation of a Bcl Xₛ pro-apoptotic splicing isoform PTM expression using a generic vector in a cancerous cell expressing the Bcl X_{L} anti-apoptotic isoform is depicted in FIGS. 1A, 1B, and 1C, and as more particularly described in detail in Example 1, infra. In yet another embodiment of the present invention, a schematic representation of a Bcl Xₛ pro-apoptotic splicing isoform PTM expression using a lentiviral vector is depicted in FIG. 2, and as more particularly described in detail in Example 2, infra. In yet another embodiment, a Bcl Xₛ pro-apoptotic splicing isoform PTM expression targeted to albumin is depicted in FIG. 3, and as more particularly described in detail in Example 3, infra.

The PTMs of the invention comprise a target binding domain that is designed to specifically bind to endogenous pre-mRNA, a 3′ splice region that includes a branch point, pyrimidine tract and a 3′ splice acceptor site and/or a 5′ splice donor site; and a spacer region that separates the RNA splice site from the target binding domain. In addition, the PTMs of the invention can be engineered to contain any nucleotide sequences encoding an apoptosis inducing splicing isoform, which upon trans-splicing, cause expression of apoptosis inducing splicing isoform or convert other highly abundant expressed proteins such as albumin to produce apoptosis inducing splicing isoform function.

In a preferred embodiment, the apoptosis inducing splicing isoform PTM translated upon trans-splicing using SMaRT™ cause expression of apoptosis inducing splicing isoform or convert other highly abundant expressed proteins such as albumin to produce apoptosis inducing splicing isoform function. The methods of the invention encompass contacting the PTMs of the invention with a natural endogenous pre-mRNA under conditions in which a portion of the PTM is trans-spliced to a portion of the natural endogenous pre-mRNA to form a novel chimeric mRNA.

The PTMs of the invention thus comprise (i) one or more target binding domains that target binding of the PTM to a pre-mRNA (ii) a 3′ splice region that includes a branch point, pyrimidine tract and a 3′ splice acceptor site and/or 5′ splice donor site; and (iii) a spacer region to separate the RNA splice site from the target binding domain. Additionally, as described above, the PTMs are engineered to contain any nucleotide sequence encoding an apoptosis inducing splicing isoform including for example, an apoptosis inducing splicing isoform gene product of a Bcl family gene, an FGFR2 family gene, p53 a family gene, an RAD51, a survivin family gene (survivin and survivin 2-B), or an Rb family gene or any combination thereof, which upon trans-splicing, cause expression of the apoptosis inducing splicing isoform or convert other abundantly expressed proteins such as albumin to produce an apoptosis inducing splicing isoform.

The target binding domain of the PTM may contain one or two binding domains of at least 15 to 30 nucleotides; or having long binding domains as described in US Patent Publication No. US 2006-0194317 A1 (the contents of which are incorporated herein by reference in their entirety), of up to several hundred nucleotides which are complementary to and in anti-sense orientation to the targeted region of the selected endogenous pre-mRNA. This confers specificity of binding and anchors the endogenous pre-mRNA closely in space so that the spliceosome processing machinery of the nucleus can trans-splice a portion of the PTM to a portion of the endogenous pre-mRNA. A second target binding region may be placed at the 3′ end of the molecule and can be incorporated into the PTM of the invention. Absolute complementarity, although preferred, is not required. A sequence “complementary” to a portion of the endogenous pre-mRNA, as referred to herein, means a sequence having sufficient complementarity to be able to hybridize with the endogenous pre-mRNA, forming a stable duplex. This is a significant advantage that the RNA trans-splicing technology of the present invention has over antisense and related splice switching oligonucleotides. The ability to hybridize will depend on both the degree of complementarity and the length of the nucleic acid (See, for example, Sambrook et al., 1989, Molecular Cloning, A Laboratory Manual, 2d Ed., Cold Spring Harbor Laboratory Press, Cold Spring Harbor, N.Y.). Generally, the longer the hybridizing nucleic acid, the more base mismatches with an RNA it may contain and still form a stable duplex. One skilled in the art can ascertain a tolerable degree of mismatch by use of standard procedures to determine the melting point of the hybridized complex. Examples of splicing isoform target specific binding domains include, for example and not by way of limitation, i) the human bcl-211 gene (Ensemble Gene ID: BCL2L1—ENSG00000171552) Bcl-2,1-rBD1: 120 bp, binding region −400 to −281 nucleotides, Ensemble transcript ID: BCL2L1-002, ENST00000376055 depicted in SEQ ID NO. 1 below;

(SEQ ID NO. 1)
TTCCAAGATA CTTCACTTAA GTCAAATCGA AAGCACCAGT
GGACTCTGAA TCTCCCACCA GCCTTTTCTA CCCCCGTCTT
CTCCGAAATG CCTTCCTCGG AAAGTCACTC CCTGGGCAGT;

the human bcl-211 gene Bcl-211-rBD2: 120 bp, binding region −530 to −411 nucleotides, Ensemble transcript ID: BCL2L1-002, ENST00000376055 depicted in SEQ ID NO. 2 below:

(SEQ ID NO. 2)
CTGAAGGGAG AGAAAGAGCT TCAGGAAAAA AAAATAATTA
ATATGCATGC CATTTACCCT AAAAATTCCA TTCCCCCTCC
AGGTACCAGA ACTGGTTTCT TTGTGGGTCT TACGAAGGTC;

the human albumin PTM target specific binding domain depicted below in SEQ ID No. 3 infra in Example 3; and ii) the mouse albumin PTM target specific binding domain depicted below in SEQ ID No. 4 infra in Example 3.

Binding may also be achieved through other mechanisms, for example, through triple helix formation or protein/nucleic acid interactions such as those in which the PTM is engineered to recognize a specific RNA binding protein, i.e., a protein bound to a specific target endogenous pre-mRNA. Alternatively, the PTMs of the invention may be designed to recognize secondary structures, such as for example, hairpin structures resulting from intramolecular base pairing between nucleotides within an RNA molecule.

The PTM molecule also contain a 3′ splice region that includes a branch point, pyrimidine tract and a 3′ splice acceptor AG site and/or a 5′ splice donor site. Consensus sequences for the 5′ splice donor site and the 3′ splice region used in RNA splicing are well known in the art (See, Moore, et al., 1993, The RNA World, Cold Spring Harbor Laboratory Press, p. 303-358). In addition, modified consensus sequences that maintain the ability to function as 5′ donor splice sites and 3′ splice regions may be used in the practice of the invention. Briefly, the 5′ splice site consensus sequence is AG/GURAGU (where A=adenosine, U=uracil, G=guanine, C=cytosine, R=purine and/=the splice site). The 3′ splice site consists of three separate sequence elements: the branch point or branch site, a polypyrimidine tract and the 3′ consensus sequence (YAG). The branch point consensus sequence in mammals is YNYURAC (Y=pyrimidine). The underlined A is the site of branch formation. A polypyrimidine tract is located between the branch point and the splice site acceptor and is important for branch point utilization and 3′ splice site recognition.

A spacer region to separate the splice sites from the target binding domain is also included in the PTM. The spacer region can have features such as stop codons which would block any translation of an unspliced PTM and/or sequences that enhance trans-splicing to the target pre-mRNA.

In a preferred embodiment of the invention, a “safety” design of the binding domain is also incorporated into the spacer, binding domain, or elsewhere in the PTM to prevent non-specific trans-splicing. The spacer sequence is a region of the PTM that covers elements of the 3′ and/or 5′ splice site of the PTM by relatively weak complementarity thereby preventing non-specific trans-splicing. The PTM is designed in such a way that upon hybridization of the binding/targeting portions of the PTM, the 3′ and/or 5′ splice site is uncovered and becomes fully active. The “safety” sequence consists of one or more complementary stretches of cis-sequence (or could be a second, separate, strand of nucleic acid) which weakly binds to one or both sides of the PTM branch point, pyrimidine tract, and/or 3′ splice site (splicing elements), or could bind to parts of the splicing elements themselves. This “safety” sequence binding prevents the splicing elements from being active (i.e., block U2 snRNP or other splicing factors from attaching to the PTM splice site recognition elements). The binding of the “safety” sequence may be disrupted by the binding of the target binding region of the PTM to the target pre-mRNA, thus exposing and activating the PTM splicing elements (making them available to trans-splice into the target endogenous pre-mRNA).

Additional features can be added to the PTM molecule either after, or before, the nucleotide sequence encoding a translatable protein, such as polyadenylation signals or 5′ splice sequences to enhance splicing, additional binding regions, “safety” sequence self-complementary regions, additional splice sites, or protective groups to modulate the stability of the molecule and prevent degradation. Additional features that may be incorporated into the PTMs of the invention include stop codons or other elements in the region between the binding domain and the splice site to prevent unspliced pre-mRNA expression. In another embodiment of the invention, PTMs can be generated with a second anti-sense binding domain downstream from the nucleotide sequences encoding a translatable protein to promote binding to the 3′ target intron or exon and to block the fixed authentic cis-5′ splice site (U5 and/or U1 binding sites). PTMs may also be made that require a double trans-splicing reaction for expression of the trans-spliced product. Such PTMs could be used to replace an internal exon which could be useful for RNA repair. Further elements such as a 3′ hairpin structure, circularized RNA, nucleotide base modification, or a synthetic analog can be incorporated into PTMs to promote or facilitate nuclear localization and spliceosomal incorporation, and intracellular stability.

The PTMs of the invention can be used in methods designed to produce a either a novel mRNA or a novel chimeric mRNA in a target cell such as, for example, a somatic cell or a germ cell. The methods of the present invention comprise delivering to the target cell a PTM which may be in any form used by one skilled in the art, for example, an RNA molecule, an RNA vector or a DNA vector which is transcribed into a RNA molecule, wherein the PTM binds to an endogenous pre-mRNA and mediates a trans-splicing reaction resulting in formation of an RNA or chimeric RNA comprising a portion of the PTM molecule spliced to a portion of the endogenous pre-mRNA.

The PTMs of the present invention can be delivered using viral vectors (e.g., lentiviral, Adeno-associated viral (“AAV”), Adenoviral, pox viral vectors, EBV, HSV, Rabies, hybrid vectors comprising AAV and Lentiviral vector, etc.) or non-viral vectors (e.g., plasmid DNA vectors including, for example, minicircle DNA vectors, (Chen et al., 2005, Hum Gene Ther 16:126-131, transposon delivery systems, phage, or PTM RNA molecules.

While the invention has been illustrated herein with the specific example of the Bcl gene, other alternatively spliced genes that are responsible for one or more diseases or disorders may be modulated by the compositions and methods of the present invention. In particular, in addition to the exemplified Bcl gene-related Bcl-x_{L} (anti-apoptotic by antagonizing and inhibiting the Bcl-2-derived proteins, Bax and Bak, induces growth of blood vessels that vascularize the tumor (angiogenesis), and promotes chemoresistance) and Bcl-xₛ (pro-apoptotic by directly binding and inhibiting or antagonizes Bcl-xL and Bcl-2 proteins, and promotes sensitization of the cancerous cells to treatment with UV- and γ-irradiation and chemotherapeutic drugs, including etoposide, 5-fluorouracil, cisplatin, 5-fluorodeoxyuridine and doxorubicin, or any combination thereof) splicing isoforms illustrated herein, other examples of alternatively spliced genes involved in the proliferation, survival and chemoresistance of cancer cells that express splice variants with different functions include, for example, and without limitation, the following: Mcl-1 gene (Mcl-1L isoform is anti-apoptotic and promotes chemoresistance; and the Mcl-1S isoform is pro-apoptotic and antagonizes Mcl-1L); Caspase-2 gene (Caspase-2L isoform is pro-apoptotic and the Caspase-2S isoform is anti-apoptotic and protects against chemotherapeutics); Caspase-9 gene (Caspase-9 isoform is pro-apoptotic and the Caspase-9β isoform is anti-apoptotic and inhibits apoptosome formation); Survivin gene (Survivin isoform is anti-apoptotic and the Survivin-2β isoform is pro-apoptotic and antagonizes survivin); Fas gene (Fas isoform mediates apoptotic signaling; and the FasExo8Del isoform inhibits Fas-mediated apoptosis and is upregulated in certain cancers); HER2 gene (HER2 isoform promotes proliferation and survival of cancer cells; the Herstatin isoform is pro-apoptotic and a soluble dominant-negative inhibitor of HER2; and the Δ15HER2 isoform is pro-apoptotic and a soluble dominant-negative inhibitor of HER2); Rac1 gene (Rac1 isoform regulates cell proliferation and cytoskeletal reorganization; and the Rac1b isoform increases the rate of GDP/GTP exchange and leads to constitutive activation, transforms cells in culture, expressed exclusively in tumor tissue); VEGF gene (VEGFA isoform promotes angiogenesis through activation of VEGF receptors 1 and 2 and is upregulated in many cancers; and the VEGF165b isoform that inhibits angiogenesis through competitive inhibition of VEGF receptor 2); p53 gene (the p53 isoform is a tumor suppressor; and the transcription factor p47 isoform that antagonizes p53 tumor suppressor); KLF6 gene (KLF6 isoform is a tumor suppressor and the transcription factor KLF6-SV1 isoform that antagonizes KLF6 and is upregulated in certain cancers); Bim gene isoforms (Bim_{L} is anti-apoptotic and Bimₛ can promote apoptosis); and those gene isoforms related to RBM5 splicing, or any combination thereof.

In addition to the cancer-related splicing isoforms illustrated supra, other examples of genes involved in the diseases or disorders that express splice variants with different functions include, for example, and without limitation, spinal muscular atrophy (SMA) SMN2 splicing, retinitis pigmentosa PRPF31 splicing, retinitis pigmentosa PRPF8 splicing, retinitis pigmentosa HPRP3 splicing, retinitis pigmentosa PAP1 splicing cartilage-hair hypoplasia (recessive), RMRP splicing, amyotrophic lateral sclerosis (ALS) TARDBP splicing, or any combination thereof.

Lentiviral Vectors

While any of a number of available vector systems may be used to express the PTMs of the present invention as described supra, what follows is a more particular description of the expression of an apoptosis inducing PTM using a lentiviral vector.

In another preferred embodiment, for each of the aforementioned compositions and methods of the present invention, the PTMs expressing the apoptosis inducing splicing isoform are introduced into the cells using, for example, certain lentiviral vector constructs including, for example, and not by way of limitation, integration competent LV, integration deficient LV, self-inactivating LV, adenovirus-LV hybrids; adeno-associated virus-LV hybrids, or any combination thereof.

In another embodiment of the present invention, the lentiviral vector of the LV-PTM of the present invention may include, without limitation, those lentiviruses can be divided into viruses that infect primate (HIV-1, HIV-2, simian immunodeficiency virus (SIV)) and non-primate (feline immunodeficiency virus (FIV), equine infectious anemia virus (EIAV), Bovine Immunodeficiency Virus (BIV), caprine arthritis encephalitis virus (CAEV), visna maedi virus (VV), Jembrana disease virus (JDV)).

In yet another aspect of the present invention, in those instances when a lentiviral vector is used for expression, the invention provides for a packaging cell line and method of making a packaging cell line for making the apoptosis inducing splicing isoform PTM constructs of the present invention. In one embodiment, a method of producing a recombinant lentiviral packaging cell is provided comprising introducing into a cell, a nucleic acid capable of expressing in said packaging cell, a nucleic acid sequence to produce transduction-competent virus-like particles; and at least one nucleic acid molecule capable of expressing the sequence of interest in said packaging cell, wherein said packaging cell produces transduction-competent virus-like particles expressing the nucleic acid sequence of interest.

In each of the aforementioned lentiviral vectors, pharmaceutical compositions containing such lentiviral vectors expressing the apoptosis inducing splicing isoform PTM constructs of the present invention, and methods of using such lentiviral vectors, the lentiviral vector further comprises one or more of the following including, for example, and not by way of limitation, a nucleic acid sequence encoding functionally active lentiviral RNA packaging elements, a nucleic acid sequence encoding functional central polypurine tract (cPPT), a central termination sequence (CTS) and 3′ LTR proximal polypurine tract (PPT), and/or a nucleic acid sequence encoding a non-protein or protein based marker or tag. In specific embodiments, the lentiviral vector of the present invention comprises one or more of the lentiviral vector constructs depicted in FIG. 2, or any combination thereof. FIG. 2 shows a non-limiting example of an LV PTM construct of the present invention.

In particular, the LV-PTM constructs of the present invention comprise a 5′ LTR and a 3′ LTR; a first nucleic acid sequence operably linked to said 5′ LTR, also referred to herein as the “payload”; and a second nucleic acid sequence, that is operably linked to said 5′ LTR wherein transcription of said first nucleic acid sequence and said second nucleic acid sequence is driven by said 5′ LTR. “Payload” is that portion of the vector that is distinct from the packaging signal required to package the RNA version of the lentiviral vector during viral production. In certain embodiments, a minimum packaging sequence may be used.

The LV-PTM vector of the present invention further comprises a nucleic acid sequence encoding functionally active lentiviral RNA packaging elements. The full-length lentiviral RNA is selectively incorporated into the viral particles as a non-covalent dimer. RNA packaging into virus particles is dependent upon specific interactions between RNA and the nucleocapsid protein (NC) domain of the Gag protein. In nature, incorporation of the HIV genomic RNA into the viral capsid (referred to as “encapsidation”) involves the so-called Psi region located immediately upstream of the Gag start codon and folded into four stem-loop structures, is important for genome packaging; SL1 to SL4. In particular, SL1 contains the dimerization initiation site (DIS), a GC-rich loop that mediates in vitro RNA dimerization through kissing-complex formation, presumably a prerequisite for virion packaging of RNA. Additional cis-acting sequences have also been shown to contribute to RNA packaging. Some of these elements are located in the first 50 nucleotides (nt) of the Gag gene, including SL4, whereas others are located upstream of the splice-donor site (SD1), and are actually mapped to a larger region covering the first 350-400 nt of the genome, including about 240 nt upstream of SL1. The SL1-4 region is an example of a simple sequence essential for RNA packaging. Other such sequences are known by those skilled in the art.

The LV-PTM constructs also comprise a nucleic acid sequence encoding a functional central polypurine tract (cPPT)/cTS and 3′ LTR proximal polypurine tract (PPT). HIV and other lentiviruses, as are known in the art, have the unique property to replicate in non-dividing cells. This property relies on the use of a nuclear import pathway enabling the viral DNA to cross the nuclear membrane of the host cell. In HIV reverse transcription, a central strand displacement event consecutive to central initiation and termination of plus strand synthesis creates a plus strand overlap; the central DNA flap. This central DNA flap is a region of triple-stranded DNA created by two discrete half-genomic fragments with a central strand displacement event controlled in cis by a central polypurine tract (cPPT) and a central termination sequence (CTS) during HIV reverse transcription. A central copy of the polypurine tract cis-active sequence (cPPT), present in all lentiviral genomes, initiates synthesis of a downstream plus strand. The upstream plus strand segment initiated at the 3′ PPT will, after a strand transfer, proceed until the center of the genome and terminate after a discrete strand displacement event. This last event of HIV reverse transcription is controlled by the central termination sequence (CTS).

In the LV-PTM vector, the transcription of the payload is driven by the 5′ LTR. The 5′ LTR has sufficient basal activity to drive transcription of a payload comprising nucleic acids that encode full length antigenic sequences, as well as packaging sequences. The 5′ LTR can be derived from various strains and clades of HIV, as are known in the art, and optimized for stronger basal promoter-like function. In particular, the 5′ LTR from HIV-1 Clade E can exhibit strong basal promoter activity. Various strains and clades of HIV are known in the art and may be used to generate the lentiviral PTM vectors of the present invention including for example, without limitation, HIV-1 groups: M (for major) (A, B, C, D, E, F, G, H, I, and J), O (outlier or “outgroup”), which is a relatively rare group currently found in Cameroon, Gabon, and France, and a third group, designated N (new group), and any circulating recombinant forms thereof. The 5′ LTR further drives expression of the payload. The HIV Rev protein directs the export of unspliced or partially spliced viral transcripts from the nucleus to the cytoplasm in mammalian cells. Rev contains the RNA binding domain, which binds the RRE present on target transcripts. Export activity is mediated by a genetically defined effector domain, which has been identified as a nuclear export signal.

The LV-PTM constructs of the present invention can comprise at least one, but can optionally comprise two or more nucleotide sequences of interest (second PTM, third PTM, etc.). In order for two or more nucleotide sequences of interest to be expressed, there may be two or more transcription units within the vector genome, one for each nucleotide sequences of interest. In those instances, it is preferable to use one or more internal ribosome entry sites (IRESs) or FMDV 2A-like sequences for translation of the second (and subsequent) coding sequence(s) in a poly-cistronic (or as used herein, “multicistronic”) message (Adam et al., 1991, J. Virol. 65:4985, the entire contents of which are incorporated herein by reference). The IRES/2A(s) may be of viral origin (such as EMCV IRES, PV IRES, or FMDV 2A-like the entire contents of which are incorporated herein by reference sequences) or cellular origin (such as, for example, and not by way of limitation, FGF2 IRES, NRF IRES, Notch 2 IRES or EIF4 IRES).

In addition, in certain embodiments of the LV-PTM constructs of the present invention, the second nucleotide sequence of interest or “payload” sequence can also include those nucleotide sequences encoding enzymes, cytokines, chemokines, growth factors, hormones, antibodies, anti-oxidant molecules, engineered immunoglobulin-like molecules, a single chain antibody, fusion proteins, immune co-stimulatory molecules, immunomodulatory molecules, a transdominant negative mutant of a target protein, a toxin, a conditional toxin, an antigen, a tumour suppresser protein and growth factors, membrane proteins, pro- and anti-angiogenic proteins and peptides, vasoactive proteins and peptides, anti-viral proteins and derivatives thereof (such as with an associated reporter group). The nucleotide sequences of interest may also encode pro-drug activating enzymes. When used in a research context, the nucleotide sequences of interest may also encode reporter genes such as, but not limited to, green fluorescent protein (GFP), luciferase, .beta.-galactosidase, or resistance genes to antibiotics such as, for example, ampicillin, neomycin, bleomycin, zeocin, chloramphenicol, hygromycin, kanamycin, among others. The nucleotide sequences of interest may also include those which function as anti-sense RNA, small interfering RNA (siRNA), or ribozymes, or any combination thereof.

In yet another embodiment of the present invention, the lentiviral vector of the present invention could be also modified by removing the transcriptional elements of HIV LTR; such as in a so-called self-inactivating (SIN) vector configuration. The modalities of reverse transcription, which generates both U3 regions of an integrated provirus from the 3′ end of the viral genome, facilitate this task by allowing the creation of so-called self-inactivating (SIN) vectors. Self-inactivation relies on the introduction of a disruption (employing for example, deletion, mutation and element insertion) in the U3 region of the 3′ long terminal repeat (LTR) of the DNA used to produce the vector RNA. During reverse transcription, this deletion is transferred to the 5′ LTR of the proviral DNA. If enough sequence is eliminated to abolish the transcriptional activity of the LTR, the production of full-length vector RNA in transduced cells is abolished. This minimizes the risk that replication competent lentiviruses (RCLs) will emerge. Furthermore, it reduces the likelihood that cellular coding sequences located adjacent to the vector integration site will be aberrantly expressed, either due to the promoter activity of the 3′ LTR or through an enhancer effect. Finally, a potential transcriptional interference between the LTR and the internal promoter driving the transgene is prevented by the SIN design. One example of a SIN based lentiviral vector is described in U.S. Pat. No. 6,924,144, the entire contents of which are incorporated herein by reference in its entirety. Non-limiting representative examples of SIN-based lentiviral vectors of the present invention may be generated from one or more of the constructs specifically shown in FIG. 2 described herein or any combination thereof.

In yet another embodiment, the lentiviral vector of the present invention could be also modified so that the left or right or both LTRs of the LV-PTM construct of the present invention contain one or more insulator element(s). Non-limiting examples of insulator sequences may be those based upon the alpha.-globin locus, including, for example, chicken HS4 such as disclosed in U.S. Patent Application Publication No. 0057725, the entire contents of which are incorporated herein by reference).

Finally, although lentiviral vectors integrate into the host genome, they can be produced as integration defective vectors by disrupting the integrase function of the HIV pol gene. This vector system will be transient in nature and vectors will be progressively lost as the cells divide thus providing an additional safety layer. Additionally, integration defective vectors will also present much lower risk of insertional mutagenesis and activation or disruption of endogenous genes.

In yet another embodiment of the present invention, the LV-PTM constructs of the present invention further comprise those lentiviral vectors in which the lentiviral integrase function has been deleted and/or abrogated by site directed mutagenesis. Insertional mutagenesis has been observed in clinical trials with oncoretroviral vectors and this has prompted detailed study of genotoxicty of all integrating vectors. The most straightforward approach for several vaccine applications would be avoiding the possibility of integration. Non-integrating lentiviral vectors have been developed by mutating the integrase gene or by modifying the attachment sequences of the LTRs. In particular, among the mutations studied, the D64V substitution in the catalytic domain has been frequently used because it shows the strong inhibition of the integrase gene without affecting proviral DNA synthesis. It has been reported that the mutation allows a transduction efficiency only slightly lower than integrative vectors but a residual integration that is about 1000-fold lower than an integrative vector at low vector doses. Another mutation described, D116N, resulted in residual integration about 2000 times lower than control vectors. In a couple of instances it has been shown that a single administration of an integrase (IN)-defective SIN LV elicits a significant immune response in the absence of vector integration and may be a safe and useful strategy for vaccine development. Thus, specifically contemplated within the scope of this invention is the modification to render the lentiviral vectors able to exist in episomal form yet still being able to provide transgene expression.

In yet another embodiment of the present invention, the LV-PTM constructs of the present invention further comprise pseudotyped lentiviral vectors. “Pseudotyping” a virion is accomplished by co-transfecting a packaging cell with both the lentiviral vector of interest and a helper vector encoding at least one envelope protein of another virus or a cell surface molecule (see, for example, U.S. Pat. No. 5,512,421, the entire text of which is herein incorporated by reference in its entirety). One viral envelope protein commonly used to pseudotype lentiviral vectors is the vesicular stomatitis virus-glycoprotein G (VSV-G), which is derived from a rhabdovirus. Other viral envelopes proteins that may be used include, for example, rabies virus-glycoprotein G and baculovirus gp-64. The use of pseudotyping broadens the host cell range of the lentiviral vector particle by including elements of the viral entry mechanism of the heterologous virus used. Pseudotyping of lentiviral vectors with, for example, VSV-G for use in the present invention results in lentiviral particles containing the lentiviral vector nucleic acid encapsulated in a nucleocapsid which is surrounded by a membrane containing the VSV-G envelope protein. The nucleocapsid preferably contains proteins normally associated with the lentiviral vector. The surrounding VSV-G protein containing membrane forms part of the viral particle upon its egress from the producer cell used to package the lentiviral vector. In an embodiment of the invention, the lentiviral particle is derived from HIV and pseudotyped with the VSV-G protein. Pseudotyped lentiviral particles containing the VSV-G protein can infect a diverse array of cell types with higher efficiency than amphotropic viral vectors. The range of host cells includes both mammalian and non-mammalian species, such as humans, rodents, fish, amphibians and insects.

Even though VSV-G pseudotyping has been described as being the most efficient for cutaneous transduction, a great advantage of using LV is that it is possible to target the vector to specific tissues or cells by replacing and/or modifying the virion envelope. LVs are remarkably compatible with a broad range of viral envelope glycoproteins providing them with added flexibility; Rabies, Mokola, LCMV, Ross River, Ebola, MuLV, Baculovirus GP64, HCV, Sindai virus F protein, Feline Endogenous Retrovirus RD114 modified, Human Endogenous Retroviruses, Seneca virus, GALV modified and HA influenza glycoproteins or a combination thereof, to name a few of those viral envelope glycoproteins explored. In addition to modification or replacement of the entire envelope, flexibility of LV platform for targeting different cell types was further demonstrated by refining the surface of LV particles via the display of cell-specific ligands. For vaccine applications, VSV-G as a pseudotyping envelope confers some important advantages, such as a broad cellular tropism (including dendritic cells) and low preexisting immunity in the human population. VSV-G could eventually be replaced by other envelopes if needed, for example in the case of multiple vector administration, although anti-VSV-G immunity does not seem to prevent repeated vector administrations.

In yet another embodiment, the invention includes a pharmaceutical composition comprising the LV-PTM construct described herein above comprising: a 5′ LTR and a 3′ LTR; a first nucleic acid sequence operably linked to said 5′ LTR; and a second nucleic acid sequence operably linked to said 5′ LTR, wherein transcription of said first nucleic acid sequence and said second nucleic acid sequence is driven by said 5′ LTR; and further comprising a “pharmaceutically acceptable carrier” or “genetic adjuvant.” “Pharmaceutically acceptable carriers” include, without limitation, PBS, buffers, water, TRIS, other isotonic solutions or any solution optimized to not damage the viral components of the vector.

The above described lentiviral vectors can be introduced into a host cell for the therapeutic treatment of diseases, as well as for other reasons described herein. Accordingly, the present invention provides a host cell comprising a vector according to the invention. The isolation of host cells, and/or the maintenance of such cells or cell lines derived therefrom in culture, has become a routine matter and one in which the ordinary skilled artisan is well versed. A “host cell” can be any cell, and, preferably, is a eukaryotic cell. Desirably, the host cell is an antigen presenting cell. Such a cell includes, but is not limited to, a skin fibroblast, a bowel epithelial cell, an endothelial cell, an epithelial cell, a dendritic cell, a plasmacytoid dendritic cell, Langerhan's cells, a monocyte, a mucosal cell and the like. Preferably, the host cell is of a eukaryotic, multicellular species (e.g., as opposed to a unicellular yeast cell), and, even more preferably, is a mammalian cell, e.g., human cell.

Thus, the present invention describes the use of SMaRT™ technology to produce, for example, apoptosis splicing isoforms or variants thereof in patient specific somatic cells or germ cells. This is achieved by trans-splicing PTMs encoding apoptosis splicing isoforms or variants thereof into one or more endogenous pre-mRNAs in somatic cells or germ cells. The target pre-mRNA transcripts can include those that are constitutively expressed or that are up or down regulated.

Alternatively, the genes or PTMs can be excised, e.g. by incorporating Lox-sites into integrating vectors and expressing Cre-recombinase, or silenced, e.g. by incorporating sequence(s) targeted by stage (lineage-, tissue-)-specific siRNA or micro-RNA, as an additional safety measure.

Methods of Use

The compositions and methods of the present invention are designed to substitute disease-causing splicing isoforms or other highly abundant expressed pre-mRNA targets, such as albumin, for example, with non-disease causing splicing isoform expression. The methods of the present invention encompass contacting a splicing isoform PTM of the invention with a natural target pre-mRNA under conditions in which all or portion of the splicing isoform PTM is spliced to the natural pre-mRNA to form a novel chimeric RNA. Generally, the target pre-mRNA is chosen because it is expressed within a specific cell type (for example, a cell type expressing the disease-causing splicing isoform) thereby providing a means for targeting expression of the novel chimeric RNA to a selected cell type, for example, and not by way of limitation, a cancer cell.

More particularly, a method is provided for inducing a non-apoptotic cell into an apoptotic cell comprising introducing into a non-apoptotic cell at least one PTM encoding a splicing isoform; trans-splicing at least one PTM encoding a splicing isoform into an endogenous pre-mRNA using SMaRT™; wherein trans-splicing of at least one PTM encoding a splicing isoform into an endogenous pre-mRNA produces a functional transcript which is then translated into a splicing isoform that induces the non-apoptotic cell into an apoptotic cell. The same effect can be achieved by splicing a PTM directly into a non-apoptotic isoform.

The method further comprises the step of target binding of said PTM, wherein the PTM comprises one or more target binding domains that target binding of the PTM to an endogenous pre-mRNA of the cell; a 3′ splice region that includes a branch point, pyrimidine tract and a 3′ splice acceptor site and/or 5′ splice donor site; a spacer region to separate the RNA splice site from the target binding domain; and a safety sequence comprising one or more complementary sequences that bind to one or both sides of the 5′ splice site, or any combination thereof.

In some embodiments of the present invention the method comprises producing a chimeric RNA molecule in a non-apoptotic cell comprising contacting a target pre-mRNA expressed in the cell with a nucleic acid molecule recognized by nuclear splicing components wherein said nucleic acid molecule comprises (a) one or more target binding domains that target binding of the nucleic acid molecule to a target pre-mRNA expressed within the cell, wherein said target binding domain targets a human albumin pre-mRNA target of the cell genome; (b) a 3′ splice region comprising a branch point and a 3′ splice acceptor site; (c) a spacer region that separates the 3′ splice region from the target binding domain; and (d) a nucleotide sequence to be trans-spliced to the target pre-mRNA wherein said nucleotide sequence encodes an apoptosis inducing splicing isoform; under conditions in which a portion of the nucleic acid molecule is trans-spliced to a portion of the target pre-mRNA to form a chimeric RNA within the cell, and wherein the splicing isoform induces the non-apoptotic cell into an apoptotic cell.

As illustrated supra, in one embodiment of the methods of the present invention, the splicing isoform targeted by one or more PTMs of the present invention comprises at least one apoptosis inducing splicing isoform gene product of a Bcl family gene, an FGFR2 family gene, p53 a family gene, an RAD51, a survivin family gene (survivin and survivin 2-B), or an Rb family gene or any combination thereof. In particular embodiments of the methods of the present invention, the apoptosis inducing splicing isoform comprises at least one of Bcl Xₛ, Mcl-1S, Caspase-2L, Caspase-9, Survivin-2B, Fas, Herstatin, Δ15HER2, Rac1, VEGF165b, p53, KLF6, and RBM5, or any combination thereof, or any combination thereof or components thereof.

In any event, regardless of which splicing isoform pre-mRNA is chosen as the target, the splicing isoform PTMs of the present invention will be expressed preferentially or exclusively in the desired target tissue by using a combination of vectors with a predilection for certain tissues, tissue-specific promoters, and/or cancer-specific promoters to achieve the desired tissue specificity. By way of illustration, tissue-specific targeted splicing isoform PTMs include, for example, and not by way of limitation, i) use of an LV vector expressing a Bcl Xs apoptosis inducing splicing isoform PTM to treat or ameliorate hepatic cancer in which the PTM expression is driven by a liver specific or tumor specific promoter; ii) use of an adeno-associated virus (AAV) vector expressing a Bcl Xs apoptosis inducing splicing isoform PTM to treat or ameliorate lung or breast cancer in which the PTM expression is driven by a combination of the cytomegalovirus (CMV) constitutive promoter and the p53 cancer-specific promoter combination; and iii) use of a plasmid or mini-circle based vector expressing an apoptosis inducing splicing isoform PTM to treat or ameliorate prostate cancer in which the PTM expression is driven by a long prostate cancer specific antigen promoter or an osteocalcin promoter.

Thus, while the PTM constructs of the present invention have been exemplified using a PTM expressing the Bcl Xₛ apoptosis inducing splice isoform, and either targeting the Bcl X_{L} pre-mRNA or specifically targeting albumin as the highly abundant pre-mRNA transcript, each of the aforementioned embodiments of the compositions and methods of the present invention can comprise PTMs that target other highly abundant or expressed pre-mRNAs such as, for example, and not by way of limitation, casein, myosin and fibroin, tumor-specific or tumor associated transcripts, microbial or autoantigen associated transcripts, viral or yeast associated transcripts, or any combination thereof. Similarly, while the LV PTM construct of the present invention has been exemplified using a PTM expressing ApoA-1, specifically targeting albumin as the highly abundant pre-mRNA transcript, the coding sequence of a protein or polypeptide of interest, for example, and not by way of limitation, that may be expressed by the PTM may include Factor VIII protein, cytokines, growth factors, insulin, hormones, enzymes, antibody polypeptides, or any combination thereof.

Pharmaceutical Compositions

The pharmaceutical compositions of the present invention contain a pharmaceutically and/or therapeutically effective amount of at least one nucleic acid construct, plasmid vector, viral vector, lentiviral vector, lentiviral vector system, viral particle/virus stock, or host cell (i.e., agents) of the invention. In one embodiment of the invention, the effective amount of an agent of the invention per unit dose is an amount sufficient to cause the detectable expression of the gene of interest. In another embodiment of the invention, the effective amount of agent per unit dose is an amount sufficient to prevent, treat or protect against deleterious effects (including severity, duration, or extent of symptoms) of the disease or condition being treated.

The administration of the pharmaceutical compositions of the invention may be for either “prophylactic” or “therapeutic” purpose. When provided prophylactically, the compositions are provided in advance of any symptom. The prophylactic administration of the composition serves to prevent or ameliorate any subsequent deleterious effects (including severity, duration, or extent of symptoms) of the disease or condition being treated. When provided therapeutically, the composition is provided at (or shortly after) the onset of a symptom of the condition being treated.

In yet another embodiment of the present invention, for all therapeutic, prophylactic and diagnostic uses, one or more of the aforementioned lentiviral vectors, lentiviral vector system, viral particle/virus stock, or host cell (i.e., agents) of the present invention, as well as other necessary reagents and appropriate devices and accessories, may be provided in kit form so as to be readily available and easily used. Such a kit would comprise a pharmaceutical composition for in vitro or in vivo administration comprising a lentiviral vector of the present invention, and a pharmaceutically acceptable carrier and/or a genetic adjuvant; and instructions for use of the kit.

The vector may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers. Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials. Extemporaneous injection solutions and suspensions may be prepared from purified nucleic acid preparations for the DNA plasmid priming compounds and/or purified viral vector compounds commonly used by one of ordinary skill in the art. Preferred unit dosage formulations are those containing a dose or unit, or an appropriate fraction thereof, of the administered ingredient. It should be understood that in addition to the ingredients, particularly mentioned above, the formulations may also include other agents commonly used by one of ordinary skill in the art.

The vector may be administered through different routes, such as oral, including buccal and sublingual, rectal, parenteral, aerosol, intranasal, intramuscular, subcutaneous, intravenous, intraperitoneal, intraocular, intracranial, intradermal, transdermal (skin patches), topical, intratumoral or direct injection into a joint or other area of the subject's body. The vector may likewise be administered in different forms, including but not limited to solutions, emulsions and suspensions, microspheres, particles, microparticles, nanoparticles, and liposomes. An appropriate quantity of LV formulation to be administered is determined by one skilled in the art based on a variety of physical characteristics of the subject or patient, including, for example, the patient's age, body mass index (weight), gender, health, immunocompetence, and the like. Similarly, the volume of administration will vary depending on the route of administration. By way of example, intramuscular injections may range from about 0.1 mL to 1.0 mL. One skilled in the art can easily determine the appropriate dose, schedule, and method of administration for the exact formulation of the composition being used, in order to achieve the desired “effective level” in the individual patient.

The vector of the present invention may be administered through various routes, including, but not limited to, oral, including buccal and sublingual, rectal parenteral, aerosol, nasal, intravenously, subcutaneous, intradermal intratumoral and topical.

The invention now being generally described, will be more readily understood by reference to the following examples, which are included merely for purposes of illustration of certain aspects and embodiments of the present invention, and are not intended to limit the invention. The contents of any patents, patent applications, patent publications, or scientific articles referenced anywhere in this application are herein incorporated in their entirety.

### EXAMPLES

### Example 1

Spliceosome mediated RNA trans-splicing (SMaRT) is one of the few RNA-based technologies that can restrict the production of a protein of therapeutic interest to a specific cell type or organ. This example involves the alternate splicing of Bcl pre-mRNA. The Bcl case illustrates how differences in trans-acting elements affect crucial differences in splice variants. Bcl has two isoforms; one of these plays a critical role in human cancers. Imbalances in these two isoforms have been implicated in several human cancers by affecting apoptosis. The anti-apoptotic Bcl-X_{L} is upregulated in several human cancers: multiple myeloma, small cell lung carcinoma, prostate and breast cancer, where it is specifically associated with an increased risk of metastasis. The pro-apoptotic Bcl-Xₛ is down regulated in transformed cells. However, forced over-expression of Bcl-XS sensitizes breast cancer cells to therapeutics.

In this example, SMaRT is used to convert Bcl-X_{L} into Bcl-X_{S}, thereby converting the tumor-associated phenotype into apoptosis associated normal phenotype, resulting in cell death. The use of SMaRT provides a powerful therapeutic approach to either address the main cause of cancer or circumvent a disease process returning to molecular normalcy.

Splicing at the downstream or upstream end of the 5′ alternate splice site of the Bcl-X produces Bcl-X_{L} or Bcl-X_{S} respectively. Bcl-X_{L} is anti-apoptotic and confers resistance to a broad variety of chemotherapeutic agents. It has also been implicated in tumor angiogenesis. Bcl-X_{S} has been shown to be pre-apoptotic and can antagonize Bcl-2 and Bcl-X_{L}.

As shown in FIG. 1, SMaRT can drive the production of Bcl-X_{S}, thereby stimulating apoptosis. There can be three different approaches for utilizing SMaRT. These include trans-splicing to a 3′ or 5′ splice site of the target pre-mRNA, or using a combination of both 3′ and 5′ splice sites, with the preferred type of trans-splicing being empirically determined by the specific cell and/or cancer. A non-limiting example of all three forms of trans-splicing is presented in FIG. 1, which schematically illustrates the use of SMaRT to mediate trans-splicing of a Bcl X splicing isoform. In particular, FIG. 1A depicts an example of 3′ exon replacement. PTM binds to intron 1 and trans-splices normal exon 2 and 3 resulting in Bcl-Xₛ pro-apoptotic mRNA (BD represents binding domain; ss represents splice site). FIG. 1B depicts an example of 5′ exon replacement wherein PTM binds to intron 2 and trans-splices normal exon 1 and 2 resulting in Bcl-Xₛ pro-apoptotic mRNA. FIG. 1C depicts an example of internal exon replacement through double trans-splicing. The double trans-splicing PTM contains two binding domains (BD1 and BD2), both 3′ and 5′ splice sites (ss), and normal exon 2. A successful double trans-splicing between the pre-mRNA target and the PTM (i.e., 5′ ss of the target pre-mRNA and 3′ ss of the PTM (r×n. #1)) followed by a successive second trans-splicing event between 5′ ss of the resulting intermediate RNA species (i.e., message) plus 3′ ss of the target (r×n. #2) results in Bcl-Xₛ pro-apoptotic mRNA.

### Example 2

This Example demonstrates the use of an LV vector expressing a PTM encoding a Bcl Xₛ apoptosis inducing splicing isoform to treat or ameliorate hepatic cancer in which the PTM expression is driven by a liver specific promoter.

While any of a number of vectors and methods may be used to express the PTMs expressing the apoptosis inducing splicing isoform, representative examples of vectors and methods of introduced the PTMs expressing the apoptosis inducing splicing isoform into the cells include, for example, and not by way of limitation, retroviral vectors, lentiviral vectors, adeno-associated viral vectors, adenoviral vectors, pox viral vectors, plasmid/minicircle vectors, viral vector transduction, electroporation, transformation, transduction, conjugation, transfection, infection, membrane fusion with cationic lipids, high-velocity bombardment with DNA-coated microprojectiles, incubation with calcium phosphate-DNA precipitate, or direct microinjection into single cells, etc.

In any event, regardless of which splicing isoform pre-mRNA is chosen as the target, the apoptosis inducing Bcl Xₛ splicing isoform PTMs will be expressed preferentially or exclusively in the desired target tissue by using a vectors with a predilection for certain tissues, and/or tissue-specific promoters to achieve the desired tissue specificity. Tissue-specific targeted splicing isoform PTMs include, for example, and not by way of limitation, i) use of an LV vector expressing a Bcl Xs apoptosis inducing splicing isoform PTM to treat or ameliorate hepatic cancer in which the PTM expression is driven by a liver specific or tumor specific promoter; ii) use of an adeno-associated virus (AAV) vector expressing a Bcl Xₛ apoptosis inducing splicing isoform PTM to treat or ameliorate lung or breast cancer in which the PTM expression is driven by a combination of the cytomegalovirus (CMV) constitutive promoter and the p53 cancer-specific promoter combination; and iii) use of a plasmid or mini-circle based vector expressing an apoptosis inducing splicing isoform PTM to treat or ameliorate prostate cancer in which the PTM expression is driven by a long prostate cancer specific antigen promoter or an osteocalcin promoter.

In this particular Example, FIG. 2 schematically illustrates a lentiviral vector expressing a Bcl X gene isoform PTM. In particular, FIG. 2A depicts a schematic diagram of a lentiviral vector expressing 3′ exon replacement PTM. FIG. 2B depicts a schematic diagram of a lentiviral vector expressing 5′ exon replacement PTM. FIG. 2C depicts a schematic diagram of a lentiviral vector expressing double trans-splicing PTM. In each instance, use of a lentiviral vector expressing a Bcl Xₛ gene isoform PTM to transduce the desired cancerous tissue of a patient with hepatic cancer results in efficient dose-dependent conversion of the Bcl-X_{L} gene isoform which is normally exhibits i) anti-apoptotic properties by antagonizing and inhibiting the Bcl-2-derived proteins, Bax and Bak, ii) induces growth of blood vessels that vascularize the tumor (angiogenesis), and iii) promotes chemoresistance) into the Bcl-X_{S} gene isoform which exhibits (pro-apoptotic by directly binding and inhibiting or antagonizes Bcl-X_{L} and Bcl-2 proteins, and promotes sensitization of the cancerous cells to treatment with UV- and γ-irradiation and chemotherapeutic drugs, including, for example, etoposide, 5-fluorouracil, cisplatin, 5-fluorodeoxyuridine and doxorubicin, or any combination thereof).

### Example 3

### Cancer Therapy—Smart-Based Strategy

This Example demonstrates that the use of SMaRT to address mechanisms of splicing aberrations has the potential to open an entirely new field of therapeutic intervention. These include those cancers involving Bcl-X_{L}: multiple myeloma, small cell lung cancer, prostate and breast cancer. In addition to Bcl pre-mRNA, targets exist for other splice isoforms involved in other human cancers. These splice isoforms including, for example, caspase 2, caspase 9, fas, HER-2, Rac-1, p53, KLF-6 and VEGF.

In particular, this Example demonstrates the specific targeting of the Bcl-X_{S} splicing isoform to an abundantly expressed target mRNA so as to achieve a high level of expression, and thereby induce a more rapid and efficient state of apoptosis in the recipient cell.

In the human plasma proteome, the protein breakdown is a1-Antitrypsin (3.8%), a2-Macroglobulin (3.6%), Immunoglobulin A (3.4%), Transferrin (3.3%), Hp Type 2-1 (2.9%), IgM (1.98%), Biomarkers (10%), and Albumin (54.3%). This provides the rationale for selecting albumin as a target for trans-splicing. Human albumin is the most abundant protein in plasma (Human: 35-50 mg/ml; Mouse: 20-30 mg/ml). Albumin is also the most abundant transcript in human liver; human liver produces 12 gms/day.

The trans-splicing into albumin approach offers several potential advantages over cDNA/recombinant protein therapy. Endogenous regulation—retains endogenous regulation of trans-spliced products, level of trans-splicing is related to level of target pre-mRNA. With SMaRT strategy described herein, the Bcl-X_{S} splicing isoform is produced in hepatocytes by inclusion of a liver specific promoter and one or more cytoplasmic targeting domains. In terms of minimized ectopic expression, trans-splicing occurs only where and when the target pre-mRNA is expressed. Endogenous protein production provides steady Bcl-X_{S} splicing isoform levels compared to high-dose/fast elimination of recombinant proteins.

Trans-Splicing the Bcl-X_{S} Splicing Isoform into Albumin

The trans-splicing of the wild type human Bcl-X_{S} splicing isoform into a highly expressed or abundant target pre-mRNA is one method of increasing the expression of human the Bcl-X_{S} splicing isoform protein. Representative examples of an endogenous highly expressed pre-mRNA molecule include, for example, albumin, casein, actin, tubulin, myosin and fibroin. Higher amounts of target pre-mRNA provide a higher trans-splicing efficiency. FIG. 3 schematically illustrates an example of targeting highly abundant transcripts such as albumin pre-mRNA target and production of Bcl-X_{S} pro-apoptotic mRNA using a 3′ PTM. PTM contains a target specific binding domain, trans-splicing domain followed by normal coding sequences (Exons 1 through 3, minus the initiation “ATG” codon). Non-limiting examples of a target specific binding domain are i) the human albumin PTM target specific binding domain depicted below in SEQ ID No. 3; and ii) the mouse albumin PTM target specific binding domain depicted below in SEQ ID No. 4.

Human Albumin Binding Domain (135 bp):

(SEQ ID No. 3)
GGTTTTATTAATAAGATAACCTTATAAGACTTCACAAATACAAAANA
CTATGCCATTTTAGAAATAAATGCCAAAATAATTCTTTAAAGATGCA
GNATTTACTAAAACTTTATTTTCCCAGTAAAATAAAGAAAC.

Mouse Albumin Binding Domain (279 bp):

(SEQ ID No. 4)
GATTCACACAACATATTTAAAGATTGATGAAGACAACTAACTGAAATA
TGCTGCTTTTTGATCTTCTCTTCACTGACCTAAGCTACTCCCTGAAGA
TGCCAGATCCCGATCGATACAGGAAAATCTGAAAAAAGCTTGCAATGG
ATCCTCTCTGCTGCACTCAAAGATATATTTTTTCACCAACATTATTAT
TTTTAAAACCCGATAAGAGATTATATCTGAGCATTCAAACTCAAGATT
TAGAGATTCTGACATGATTGAAAATATCTACTAAGAAAA.

Upon administration of the human Bcl-X_{S} splicing isoform PTM (for example, using a lentiviral viral-based expression vector using a liver tissue specific or tumor specific promoter) to the patient having an advanced case of non-metastasized hepatocellular carcinoma, trans-splicing to the albumin target pre-mRNA occurs and the Bcl-X_{S} splicing isoform PTM acquires the ATG initiation codon resulting in a functional trans-spliced chimeric pro-apoptotic mRNA which upon translation, processing and secretion produces functional chimeric pro-apoptotic Bcl-X_{S} protein. As a result of the trans-splicing reaction, a chimeric albumin-Bcl-X_{S} gene isoform fusion protein is produced which exhibits pro-apoptotic activity by directly binding and inhibiting or antagonizing Bcl-X_{L} and Bcl-2 proteins, thereby resulting in reduction of the tumor load or burden in the patient's advanced case of non-metastasized hepatocellular carcinoma.

In addition, because of the upregulation and increased expression of the chimeric albumin-Bcl-X_{S} gene isoform fusion protein promotes sensitization of the cancerous cells to treatment with UV- and γ-irradiation and chemotherapeutic drugs, the concomitant or subsequent treatment of the patient's advanced case of non-metastasized hepatocellular carcinoma with a UV- and γ-irradiation and/or one or more chemotherapeutic drugs, including, for example, etoposide, 5-fluorouracil, cisplatin, 5-fluorodeoxyuridine and doxorubicin (or a combination thereof) results in dose-dependent reduction of the tumor load or burden in the patient's advanced case of non-metastasized hepatocellular carcinoma.

Having now described a few embodiments of the invention, it should be apparent to those skilled in the art that the foregoing is merely illustrative and not limiting, having been presented by way of example only. Numerous modifications and other embodiments are within the scope of one of ordinary skill in the art and are contemplated as falling within the scope of the invention and any equivalent thereto. It can be appreciated that variations to the present invention would be readily apparent to those skilled in the art, and the present invention is intended to include those alternatives. Further, because numerous modifications will readily occur to those skilled in the art, it is not desired to limit the invention to the exact construction and operation illustrated and described, and accordingly, all suitable modifications and equivalents may be resorted to, falling within the scope of the invention.

What is claimed is:

1. An isolated non-apoptotic cell, comprising:
at least one pre-trans-splicing molecule (PTM),
whereby upon trans-splicing using spliceosome-mediated RNA trans-splicing (SMaRT), said isolated non-apoptotic cell produces a splicing isoform that drives said non-apoptotic cell into apoptosis.

2. The cell of claim 1, wherein said PTM further comprises:
(a) one or more target binding domains that targets binding of the PTM to an endogenous pre-mRNA;
(b) a 3′ splice region that includes a branch point, pyrimidine tract, and either a 3′ splice acceptor site and a 5′ splice donor site or a 5′ splice donor site;
(c) a spacer region to separate the RNA splice site from the target binding domain; and
(d) a safety sequence comprising one or more complementary sequences that bind to one or both sides of the 5′ splice site, or any combination thereof.

3. The cell of claim 1, said splicing isoform comprising at least one apoptosis inducing splicing isoform.

4. The cell of claim 3, said apoptosis inducing splicing isoform comprising an apoptosis inducing splicing isoform gene product of a Bcl family gene, an FGFR2 family gene, p53 a family gene, an RAD51, a survivin family gene (survivin and survivin 2-B), or an Rb family gene or any combination thereof.

5. The cell of claim 4, said apoptosis inducing splicing isoform comprising at least one of Bcl Xs, Mcl-1S, Caspase-2L, Caspase-9, Survivin-2B, Fas, Herstatin, Δ15HER2, Rac1, VEGF165b, p53, KLF6, and RBM5, or any combination thereof.

6. The cell of claim 4, wherein said cell is a cancer cell expressing Bcl X_{L} or a functional derivative thereof.

7. The cell of claim 6, wherein said cancer cell is selected from the group consisting of multiple myeloma, non-small cell lung cancer, prostate, breast cancer, glioma, large intestinal cancer, lung cancer, small cell lung cancer, stomach cancer, liver cancer, blood cancer, bone cancer, pancreatic cancer, skin cancer, head or neck cancer, cutaneous or intraocular melanoma, uterine sarcoma, ovarian cancer, rectal or colorectal cancer, anal cancer, colon cancer, fallopian tube carcinoma, endometrial carcinoma, cervical cancer, vulval cancer, squamous cell carcinoma, vaginal carcinoma, Hodgkin's disease, non-Hodgkin's lymphoma, multiple myeloma, esophageal cancer, small intestine cancer, endocrine cancer, thyroid cancer, parathyroid cancer, adrenal cancer, soft tissue tumor, urethral cancer, penile cancer, prostate cancer, chronic or acute leukemia, lymphocytic lymphoma, bladder cancer, kidney cancer, ureter cancer, renal cell carcinoma, renal pelvic carcinoma, CNS tumor, glioma, astrocytoma, glioblastoma multiforme, primary CNS lymphoma, bone marrow tumor, brain stem nerve gliomas, pituitary adenoma, uveal melanoma, testicular cancer, oral cancer, pharyngeal cancer, pediatric neoplasms, leukemia, neuroblastoma, retinoblastoma, rhabdomyoblastoma and sarcoma.

8. A method of inducing a non-apoptotic cell into an apoptotic cell comprising:
(a) introducing into said non-apoptotic cell at least one pre-trans-splicing molecule (PTM) encoding a splicing isoform; and
(b) trans-splicing said at least one PTM encoding a splicing isoform into an endogenous pre-mRNA using spliceosome-mediated RNA trans-splicing (SMaRT), whereby trans-splicing of at least one PTM encoding a splicing isoform into an endogenous pre-mRNA produces a functional transcript which is then translated into a splicing isoform that drives the non-apoptotic cell into apoptosis.

9. The method of claim 8, further comprising the step of targeting the binding of said PTM, said PTM comprising:
(a) one or more target binding domains that targets binding of the PTM to an endogenous pre-mRNA of the cell;
(b) a 3′ splice region that includes a branch point, pyrimidine tract and either a 3′ splice acceptor site and a 5′ splice donor site or a 5′ splice donor site;
(c) a spacer region to separate the RNA splice site from the target binding domain; and
(d) a safety sequence comprising one or more complementary sequences that bind to one or both sides of the 5′ splice site, or any combination thereof.

10. The method of claim 8, said splicing isoform comprising at least one apoptosis inducing splicing isoform.

11. The method of claim 8, said apoptosis inducing splicing isoform comprising an apoptosis inducing splicing isoform gene product of a Bcl family gene, an FGFR2 family gene, p53 a family gene, an RAD51, a survivin family gene (survivin and survivin 2-B), or an Rb family gene or any combination thereof.

12. The method of claim 8, said apoptosis inducing splicing isoform comprising at least one of Bcl Xs, Mcl-1S, Caspase-2L, Caspase-9, Survivin-2B, Fas, Herstatin, Δ15HER2, Rac1, VEGF165b, p53, KLF6, and RBM5, or any combination thereof.

13. The method of claim 8, wherein said cell is a cancer cell expressing Bcl X_{L} or a functional derivative thereof.

14. The method of claim 13, wherein said cancer cell is selected from the group consisting of multiple myeloma, non-small cell lung cancer, prostate and breast cancer said cancer is selected from the group consisting of breast, glioma, large intestinal cancer, lung cancer, small cell lung cancer, stomach cancer, liver cancer, blood cancer, bone cancer, pancreatic cancer, skin cancer, head and neck cancer, cutaneous or intraocular melanoma, uterine sarcoma, ovarian cancer, rectal or colorectal cancer, anal cancer, colon cancer, fallopian tube carcinoma, endometrial carcinoma, cervical cancer, vulval cancer, squamous cell carcinoma, vaginal carcinoma, Hodgkin's disease, non-Hodgkin's lymphoma, multiple myeloma, esophageal cancer, small intestine cancer, endocrine cancer, thyroid cancer, parathyroid cancer, adrenal cancer, soft tissue tumor, urethral cancer, penile cancer, prostate cancer, chronic or acute leukemia, lymphocytic lymphoma, bladder cancer, kidney cancer, ureter cancer, renal cell carcinoma, renal pelvic carcinoma, CNS tumor, glioma, astrocytoma, glioblastoma multiforme, primary CNS lymphoma, bone marrow tumor, brain stem nerve gliomas, pituitary adenoma, uveal melanoma, testicular cancer, oral cancer, pharyngeal cancer, pediatric neoplasms, leukemia, neuroblastoma, retinoblastoma, glioma, rhabdomyoblastoma and sarcoma.

15. A method of producing a chimeric RNA molecule in a non-apoptotic cell comprising: contacting a target pre-mRNA expressed in the cell with a nucleic acid molecule recognized by nuclear splicing components wherein said nucleic acid molecule comprises:
(a) one or more target binding domains that target binding of the nucleic acid molecule to a target pre-mRNA expressed within the cell, wherein said target binding domain targets a human albumin pre-mRNA target of the cell genome;
(b) a 3′ splice region comprising a branch point and a 3′ splice acceptor site;
(c) a spacer region that separates the 3′ splice region from the target binding domain; and
(d) a nucleotide sequence to be trans-spliced to the target pre-mRNA
wherein said nucleotide sequence encodes an apoptosis inducing splicing isoform; under conditions in which a portion of the nucleic acid molecule is trans-spliced to a portion of the target pre-mRNA to form a chimeric RNA within the cell, and wherein the splicing isoform drives the non-apoptotic cell into apoptosis.

16. The method according to claim 15, wherein said non-apoptotic cell expresses Bcl X_{L} or a functional derivative thereof.

17. The method according to claim 15, wherein said non-apoptotic cell either contains no Bcl X_{L} nucleotide sequences or expresses no Bcl X_{L} protein or functional derivative thereof.

18. A nucleic acid molecule comprising:
a) one or more target binding domains that target binding of said nucleic acid molecule to an apoptosis inducing splicing isoform target pre-mRNA expressed within a cell;
b) a 5′ splice site;
c) a spacer region that separates the 5′ splice site from the target binding domain;
d) a safety sequence comprising one or more complementary sequences that bind to one or both sides of the 5′ splice site; and
e) a nucleotide sequence to be trans-spliced to said target pre-mRNA,
wherein said nucleic acid molecule is recognized by nuclear splicing components within said cell.

19. A nucleic acid molecule comprising:
a) one or more target binding domains that target binding of the nucleic acid molecule to an apoptosis inducing splicing isoform target pre-mRNA expressed within a cell;
b) a 3′ splice region comprising a branch point, a pyrimidine tract, and a 3′ splice acceptor site;
c) a spacer region that separates the 3′ splice region from the target binding domain;
d) a safety sequence comprising one or more complementary sequences that bind to one or both sides of the 3′ splice site; and
e) a nucleotide sequence to be trans-spliced to the target pre-mRNA,
wherein said nucleic acid molecule is recognized by nuclear splicing components within said cell.

20. A eukaryotic expression vector wherein said vector expresses a nucleic acid molecule comprising:
a) one or more target binding domains that target binding of the nucleic acid molecule to an apoptosis inducing splicing isoform target pre-mRNA expressed within a cell;
b) a 3′ splice region comprising a branch point, a pyrimidine tract, and a 3′ splice acceptor site;
c) a spacer region that separates the 3′ splice region from the target binding domain; and
d) a nucleotide sequence to be trans-spliced to the target pre-mRNA,
wherein said nucleic acid molecule is recognized by nuclear splicing components within the cell.

21. A eukaryotic expression vector wherein said vector expresses a nucleic acid molecule comprising:
a) one or more target binding domains that target binding of the nucleic acid molecule to an apoptosis inducing splicing isoform target pre-mRNA expressed within a cell;
b) a 5′ splice site;
c) a spacer region that separates the 5′ splice site from the target binding domain; and
d) a nucleotide sequence to be trans-spliced to the target pre-mRNA,
wherein said nucleic acid molecule is recognized by nuclear splicing components within the cell.

22. A composition comprising a physiologically acceptable carrier and the nucleic molecule of claim 18.

23. A composition comprising a physiologically acceptable carrier and the nucleic molecule of claim 19.


SEQUENCE LISTING (4 sequences)

SEQ ID NO: 1 - DNA, 120 bp, Homo sapiens
ttccaagatacttcacttaagtcaaatcgaaagcaccagtggactctgaatctcccaccagccttttctacccccgtcttctccgaaatgccttcctcggaaagtcactccctgggcagt

SEQ ID NO: 2 - DNA, 120 bp, Homo sapiens
ctgaagggagagaaagagcttcaggaaaaaaaaataattaatatgcatgccatttaccctaaaaattccattccccctccaggtaccagaactggtttctttgtgggtcttacgaaggtc

SEQ ID NO: 3 - DNA, 135 bp, Homo sapiens
  misc_feature (46)..(46): n is a, c, g, or t
ggttttattaataagataaccttataagacttcacaaatacaaaanactatgccattttagaaataaatgccaaaataattctttaaagatgcagnatttactaaaactttattttcccagtaaaataaagaaac

SEQ ID NO: 4 - DNA, 279 bp, Mus musculus
gattcacacaacatatttaaagattgatgaagacaactaactgaaatatgctgctttttgatcttctcttcactgacctaagctactccctgaagatgccagatcccgatcgatacaggaaaatctgaaaaaagcttgcaatggatcctctctgctgcactcaaagatatattttttcaccaacattattatttttaaaacccgataagagattatatctgagcattcaaactcaagatttagagattctgacatgattgaaaatatctactaagaaaa